SEQUENCE LISTING (2 sequences)

SEQ ID NO: 1 - DNA, 36 bp, synthetic construct
  misc_feature 1..36: note = synthetic construct
  misc_difference 1..6: note = n is a, c, g, t, or u
  source 1..36: mol_type = other DNA
nnnnnngtcgacatggtcttcacactcgaagatttc

SEQ ID NO: 2 - DNA, 34 bp, synthetic construct
  misc_feature 1..34: note = synthetic construct
  misc_difference 1..6: note = n is a, c, g, t, or u
  source 1..34: mol_type = other DNA
nnnnnnccgcggttacgccagaatgcgttcgcac

(19) United States
(12) Patent Application Publication
(10) Pub. No.: US 2025/0332170 A1
(43) Pub. Date: Oct. 30, 2025

(54) VANDETANIB REDUCES INFLAMMATORY CYTOKINES AND AMELIORATES COVID-19

(71) Applicant: Collaborations Pharmaceuticals, Inc., Fuquay Varina, NC (US)

(72) Inventors: Ana C. Puhl, Fuquay Varina, NC (US); Sean Ekins, Fuquay Varina, NC (US)

(73) Assignee: Collaborations Pharmaceuticals, Inc., Fuquay Varina, NC (US)

(21) Appl. No.: 18/720,768

(22) PCT Filed: Dec. 15, 2022
(86) PCT No.: PCT/US2022/081716

§ 371 Date: Jun. 17, 2024

Related U.S. Application Data
(60) Provisional application No. 63/290,051, filed on Dec. 15, 2021.

(51) Int. Cl. A61K 31/517; A61P 31/14

(52) U.S. Cl. CPC A61K 31/517; A61P 31/14

(57) ABSTRACT

Compositions for and methods of treating lung conditions, such as those associated with elevated cytokines, are described. The methods include administration of vandetanib or a pharmaceutically acceptable salt thereof. Methods of treating COVID-19 via administration of vandetanib or a pharmaceutically acceptable salt thereof are also described. Administration of vandetanib can reduce levels of cytokines that are elevated in subjects suffering from COVID-19 and mitigate or eliminate the cytokine storm associated with severe cases of COVID-19.

### CROSS-REFERENCE TO RELATED APPLICATIONS

This application is a U.S. National Phase application of PCT International Patent Application No. PCT/US2022/081716, filed Dec. 15, 2022, incorporated herein by reference in its entirety, and which claims priority to and benefit of U.S. Provisional Patent Application Ser. No. 63/290,051, filed Dec. 15, 2021, which is herein incorporated by reference in its entirety.

### GOVERNMENT SUPPORT

This invention was made with government support under Grant Number IR43AT010585-01 awarded by the National Institutes of Health. The government has certain rights in the invention.

### REFERENCE TO SEQUENCE LISTING XML SUBMITTED ELECTRONICALLY

The content of the Sequence Listing XML filed using Patent Center as an XML file (Name: Replacement_3270_21_PCT.xml; Size: 4,471 bytes; and Date of Creation: Dec. 18, 2024) is incorporated herein by reference in its entirety.

### TECHNICAL FIELD

The presently disclosed subject matter relates to methods of treating COVID-19 and other lung conditions related to elevated inflammatory cytokines via administration of vandetanib or pharmaceutically acceptable salts thereof.

### ABBREVIATIONS

%=percent
μL=microliter
μm=micrometer
μM=micromolar
ACE2=angiotensin converting enzyme 2
ARDS=acute respiratory disease syndrome
avg=average
BMP=bis(monoacylglycerol)phosphate
CC₅₀=50% cytotoxic concentration
CCL=chemokine (C—C motif) ligand
COVID-19=coronavirus disease
CXCL=chemokine (C—X—C motif) ligand
DBT=delayed brain tumor
DMSO=dimethyl sulfoxide
EC₉₀=90% effective concentration
EGFR=epidermal growth factor receptor
ELISA=enzyme-linked immunosorbent assay
h=hours
IC₅₀=50% inhibitory concentration
IFN=interferon
IL=interleukin
i.p.=intraperitoneal
kg=kilogram
MERS-CoV=Middle East respiratory syndrome coronavirus
mg=milligram
MHV=murine hepatitis virus
mL=milliliter
MODS multi-organ dysfunction syndrome
MOI=multiplicity of infection
MST=microscale thermophoresis
PFU=plaque-forming unit
pg=picogram
POC=percentage of control
RT-PCR=reverse transcription-polymerase chain reaction
SARS-CoV-2=severe acute respiratory coronavirus 2
SI=selectivity index
TCID50=median tissue culture infectious dose
TNFα=tumor necrosis factor alpha
VEGFR=vascular endothelial growth factor

### BACKGROUND

Currently three vaccines am approved for SARS-CoV-2 in the USA.¹⁻³ In contrast, there are relatively few small-molecule drugs that are approved for use. These include remdesivir,⁴ while molnupiravir is approved in the United Kingdom and has an emergency use authorization in the US. Another drug, the protease inhibitor Nirmatrelvir/ritonavir, also has an emergency use authorization from the FDA. An emergency use authorization allows the protein kinase inhibitor baricitinib to be combined with remdesivir in hospitalized adults and children 2 years and older who require respiratory support.⁵ The National Institutes of Health COVID-19 treatment guidelines recommend the use of dexamethasone in certain patients hospitalized with severe COVID-19 based on results from the RECOVERY trial.⁶

As these limited treatment options and several drugs in clinical trials⁷ attest, there is a ongoing need for additional therapeutic options for treating COVID-19. In particular, there is a need for orally available small molecule therapeutic treatment options that can be used outside of hospitals and for treatments to address the many symptoms of COVID-19 that are termed long-COVID.^{8,9}

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter provides a method of treating a lung condition in a subject in need of treatment thereof, the method comprising administering to the subject a therapeutically effective amount of vandetanib or a pharmaceutically acceptable salt thereof. In some embodiments, the lung condition is characterized by a level of one or more cytokines that are elevated in the subject compared to a subject that does not have the lung condition. In some embodiments, the lung condition is selected from the group comprising coronavirus disease (COVID-19), bronchopulmonary dysplasia, pulmonary fibrosis, and chemical exposure.

In some embodiments, the subject is a mammalian subject. In some embodiments, the subject is a human subject.

In some embodiments, the administration reduces a level of one or more of interleukin-6 (IL-6), interleukin-10 (IL-10), and tumor necrosis factor alpha (TNF-α) in the subject. In some embodiments, the administration increases a level of interferon 1β (IFN-1β) in the subject. In some embodiments, the administration reduces a level of one or more chemokine (C—C motif) ligand (CCL) in the subject. In some embodiments, the one or more CCL is selected from the group comprising CCL2, CCL3, and CCKL4.

In some embodiments, the presently disclosed subject matter provides a method of treating COVID-19 in a subject in need of treatment thereof, the method comprising administering to the subject a therapeutically effective amount of vandetanib or a pharmaceutically acceptable salt thereof. In some embodiments, the COVID-19 is characterized by a level of one or more cytokines that are elevated in the subject compared to a subject that does not have the lung condition.

In some embodiments, the subject is a mammalian subject. In some embodiments, the subject is a human subject. In some embodiments, the subject is a hospitalized subject and/or a subject receiving supplemental oxygen.

In some embodiments, the administration reduces a level of one or more of IL-6, IL-10, and TNF-α in the subject. In some embodiments, the administration increases a level of IFN-β in the subject. In some embodiments, the administration reduces a level of one or more CCL in the subject. In some embodiments, the one or more CCL is selected from the group comprising CCL2, CCL3, and CCL4.

In some embodiments, the presently disclosed subject matter provides a composition for use in treating a lung condition in a subject in need of treatment thereof, the composition comprising a therapeutically effective amount of vandetanib or a pharmaceutically acceptable salt thereof. In some embodiments, the lung condition is characterized by a level of one or more cytokines that are elevated in the subject compared to a subject that does not have the lung condition, optionally wherein the lung condition is selected from the group consisting of COVID-19, bronchopulmonary dysplasia, pulmonary fibrosis, and chemical exposure.

In some embodiments, the presently disclosed subject matter provides a composition for use in treating COVID-19 in a subject in need of treatment thereof, the composition comprising a therapeutically effective amount of vandetanib or a pharmaceutically acceptable salt thereof.

Accordingly, it is an object of the presently disclosed subject matter to provide methods of treating lung conditions and methods of treating COVID-19 that comprise administering vandetanib or a pharmaceutically acceptable salt thereof, and related compositions for use in treating lung conditions (e.g., COVID-19).

An object of the presently disclosed subject matter having been stated hereinabove, and which is achieved in whole or in part by the presently disclosed subject matter, other objects will become evident as the description proceeds hereinbelow.

### BRIEF DESCRIPTION OF THE FIGURES

FIGS. 1A-1G. FIGS. 1A-1C are graphs showing severe acute respiratory syndrome coronavirus 2 (SARS-CoV-2) inhibition (percent inhibition versus compound dose expressed as the log value of the micromolar (μM) concentration) in A549-ACE2 cell lines and cytotoxicity (percent versus compound dose) of (FIG. 1A) remdesivir (FIG. 1B) entrectinib, and (FIG. 1C) vandetanib. FIGS. 1D and 1E are graphs showing the results of HCoV229E antiviral assay with (FIG. 1D) entrectinib and (FIG. 1E) vandetanib. Results are shown as the log of the median tissue culture infectious dose (TCID₅₀) per milliliter (mL) for different concentrations of compound (0 μM-50 μM) FIG. 1F is a series of images of a pseudo SARS-CoV-2 D614G baculovirus assay without virus or vandetanib (top), with virus but without vandetanib (middle) or with virus and in the presence of vandetanib at 1 μM (bottom). FIG. 1 G is a graph showing analysis of the assay results (spot total count) shown in FIG. 1F.

FIGS. 2A-2E: In vivo efficacy of vandetanib in a mouse model of coronavirus disease (COVID-19). FIG. 2A is a schematic diagram showing the experimental timeline: K18-hAce2 tg mice were infected with severe acute respiratory syndrome coronavirus 2 (SARS-CoV-2) (2×10⁴ plaque-forming units per 40 microliters (PFU/40 μL) saline, intranasal) or mock. One group of mice was treated with Vandetanib (25 milligrams per kilogram (mg/kg) intraperitoneal (i.p.) injection) 1 hour (h) before virus inoculation. FIG. 2B is a graph showing the body weight (expressed as a percentage of the baseline) in the mock group (circles), infected group (squares) and infected and treated (diamonds) evaluated daily. FIG. 2C is a graph of the lung viral load evaluated in mice euthanized at three days post infection (DPI), expressed as a log of the number of viral copies. FIG. 2D is a graph of inflammation in the lungs of mice euthanized at three DPI (expressed as an area fraction). FIG. 2E is a series of micrographs (20 times magnification (20×) on left, 40 times magnification (40×) on right) of the lung histopathology of the different groups of mice at three DP1. *** p<0.001 as compared with mock group after one-way ANOVA followed by Tukey post-hoc test. ###p<0.001 as compared with infected group after one-way ANOVA followed by Tukey post-hoc test. The scale bar for the 20× images represents 125 micrometers (μm). The scale bar for the 40× images represents 50 μm.

FIG. 3A-3K: A series of graphs showing the effects of vandetanib on severe acute respiratory syndrome coronavirus 2 (SARS-CoV-2)-induced lung inflammation in a mouse model of coronavirus disease (COVID-19). Data in each graph is shown for three groups of mice: a control group that was untreated and not infected with SARS-CoV-2 as described for FIGS. 2A-2E (Mock, circles), a group that was infected with SARS-CoV-2 as described for FIGS. 2A-2E (Infected, squares), and a group that was infected with SARS-CoV-2 and treated with vandetanib as described for FIGS. 2A-2E (infected+Van, diamonds). FIG. 3A shows the expression of interferon 1-beta (IFN-1β) quantified by quantitative polymerase chain reaction (qPCR). Levels of (FIG. 3B) IFN-1β, (FIG. 3C) interleukin 6 (IL-6), (FIG. 3D) tumor necrosis factor-alpha (TNF-α), (FIG. 3E) chemokine (C—C motif) ligand 4 (CCL4), (FIG. 3F) chemokine (C—C motif) ligand 2 (CCL2), (FIG. 3G) chemokine (C—C motif) ligand 3 (CCL3), (FIG. 3H) interleukin 10 (IL-10), (FIG. 3I) chemokine (C—X—C motif) ligand 1 (CXCL1), (FIG. 3J) chemokine (C—X—C motif) ligand 2 (CXCL2), and (FIG. 3K) chemokine (C—X—C motif) ligand 10 (CXCL10) measured by enzyme-linked immunosorbent assay (ELISA). * p<0.05, ** p<0.01, and *** p<0.001 as compared with mock group afterone-way ANOVA followed by Tukey post-hoc test. #p<0.05 and ##p<0.01 as compared with infected group after one-way ANOVA followed by Tukey post-hoc test.

FIGS. 4A and 4B: Graphs showing in vino antiviral severed acute respiratory syndrome coronavirus 2 (SARS-CoV-2) testing of (FIG. 4A) remdesivir and (FIG. 4B) entrectinib in Calu-3 cells. Calu-3 (ATCC, HTB-55) cells were pretreated with remdesivir or entrectinib for 2 hours prior to continuous infection with SARS-CoV-2 (isolate USA WA1/2020) at a multiplicity of infection (MOI)=0.5. Forty-eight hours post-infection, cells were fixed, immunostained, and imaged by automated microscopy for infection (double stranded RNA (dsRNA)+ cells/total cell number) and cell number. Sample well data was normalized to aggregated dimethyl sulfoxide (DMSO) control wells and plotted versus drug concentration to determine the 50 percent inhibitory concentration (IC₅₀; infection: squares) and 50 percent cytotoxic concentration (CC₅₀ toxicity: circles). Percentage of Control (POC)=(sample well measurement/aggregated DMSO average (avg))*100 for n=3 replicates.

FIG. 5 is a graph showing in vitro activity of vandetanib against murine hepatitis virus (MHV) in delayed brain tumor (DBT) cells, a model for severe acute respiratory syndrome coronavirus 2 (SARS-CoV-2) replication. The results are shown as a percent (%) virus inhibition compared to dimethyl sulfoxide (DMSO) treated control cells versus the log of vandetanib concentration (molar (M)). The 50% inhibitory concentration (IC₅₀) for vandetanib in this model is 1.60 micromolar (μM).

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### 1. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a”, “an”, and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising”, which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

Unless otherwise indicated, all numbers expressing quantities of time, concentration, dosage and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value is meant to encompass variations of in one example ±20% or ±10%, in another example ±5%, in another example ±1%, and in still another example ±0.1% from the specified amount, as such variations are appropriate to perform the disclosed methods.

As use herein, the terms “administration of” and/or “administering” a compound can be understood to refer to providing a compound of the presently disclosed subject matter to a subject in need of treatment. As used herein “administering” includes administration of a compound or compounds by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

The term “cytokine” as used herein refers to small (e.g., 5-25 kiloDalton) proteins involved in cell signaling, e.g., with regard to modulation of immune responses. Cytokines act through cell surface receptors and are produced by immune cells, as well as some other cells, such as endothelial cells. A number of families of cytokines have been characterized, including, but not limited to, chemokines, interferons, interleukins, lymphokines, and tumor necrosis factors.

“Chemokine,” as used herein, refers to an intercellular signaling molecule involved in the chemotaxis.

As used herein, the term “pharmaceutically acceptable carrier” means a composition with which an appropriate compound can be combined and which, following the combination, can be used to administer the appropriate compound to a subject.

As used herein, the term “pharmaceutically acceptable” ester or salt means an ester or salt form of the active ingredient which is compatible with any other ingredients of the pharmaceutical composition, which is not deleterious to the subject to which the composition is to be administered.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, can be different from when that compound is administered alone. Thus, an effective amount of a combination of compounds refers collectively to the combination as a whole, although the actual amounts of each compound can vary. The term “more effective” means that the selected effect occurs to a greater extent by one treatment relative to the second treatment to which it is being compared.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

### II. General Considerations

Drug repurposing is a strategy that can accelerate the discovery of small molecule treatments for COVID-19, e.g., by providing expedited clinical progression.^{10,11} Repurposing efforts have already identified molecules (such as remdesivir) originally developed for other viruses and approved outside the USA with potent in vitro activity against SARS-CoV-2.¹² Small to medium scale assays and high-throughput screening (HTS) campaigns¹³ have been performed for testing FDA-approved drugs. The catalytic activity of the viral targets main protease (M^{pre}) and papain-like protease (PL^{pro}) are involved in viral replication, making inhibition of these enzymes a compelling strategy for antiviral therapy.¹⁴ The discovery of PF-008352313 as a covalent active-site-directed inhibitor of SARS-CoV M^{pro} in 2003 provided for the translation of this agent into clinical trials for SARS-CoV-2 in 2202.¹⁵ Pfizer also developed the SARS-CoV-2 inhibitor PF-07321332 targeting M^{pro}, in combination with ritonavir. This combination is sold under the tradename PAXLOVID® (Pfizer Inc., New York, New York, United States of America), and an interim analysis of the Phase 2/3 EPIC-HR study showed it reduced risk of death or hospitalization by 89%. However, all of these direct acting antivirals target early-stage virus replication and have a short therapeutic window, which would render them less effective if administered during the immunopathogenic phase of the disease.

When SARS-CoV-2 invades the body, it can cause an imbalance in the immune system that can result in a cytokine storm.¹⁶ COVID-19 patients can deteriorate over a short period, leading to acute respiratory distress syndrome (ARDS), coagulation disorders, and eventually multiple organ failure.^{16,17} COVID-19 displays an “inflammatory signature”, characterized by increased levels of soluble biomarkers (cytokine and chemokines), which are involved in the recruitment and activation of several immune cell types, like monocyte/macrophages, neutrophils, T-lymphocytes, and many others.¹⁸ These immune-active biomarkers, measured either early upon patient admission or throughout hospitalization, can provide clinically relevant information in predicting a more or less severe course of the disease, as well as in estimating the mortality risk of infected patients.¹⁹ Targeting the cytokine storm to ameliorate the state of hyperinflammation has potential as a novel therapeutic approach in the treatment of COVID-19.^{18,20-22} Providing more targeted therapeutic approaches could allow for an earlier anti-cytokine treatment and prevention of ARDS and/or death. It was previously demonstrated that the modulation of host cell signaling is important for viral replication in many viruses and could have therapeutic relevance^{23,24} To date, the FDA has approved over 60 small molecule protein kinase inhibitors and most of these are used in the treatment of cancers (e.g. leukemias, breast and lung cancers) whereas several are for non-malignancies.²⁵

### III. Representative Methods of The Presently Disclosed Subject Matter

As described hereinbelow, forty-five (45) FDA-approved protein kinase inhibitors were tested in vitro against murine hepatitis virus (MIV) as a model of SARS-CoV-2 replication. Twelve (12) showed inhibition of MHV in the delayed brain tumor (DBT) cell line. Based on the results, two compounds, entrectinib and vandetanib, were evaluated in A549-ACE2 cells infected by SARS-CoV-2. Vandetanib, which targets the vascular endothelial growth factor receptor (VEGFR), the epidermal growth factor receptor (EGFR), and the RET-tyrosine kinase showed the most promising results on inhibition versus toxic effect on SARS-CoV-2-infected Caco-2 and A549-hACE2 cells (IC₅₀ 0.79 μM), while also showing a reduction of >3 log TCID50/ml for HCoV-229E.

In addition, the in vivo efficacy of vandetanib was evaluated in an acute infection model using K-18-hACE2 mice challenged with SARS-CoV-2. Vandetanib statistically significantly reduced the levels of IL-6, IL-10, TNF-α, and mitigated inflammatory cell infiltrates in the lungs of infected animals but did not reduce viral load. Further, vandetanib rescued the decreased IFN-1β caused by SARS-CoV-2 infection in mice to levels similar to that in uninfected animals.

Thus, according to one aspect, the presently disclosed subject matter provides a method of treating a lung condition in a subject in need of treatment thereof. In some embodiments, the method comprises administering to the subject a therapeutically effective amount of vandetanib or a pharmaceutically acceptable salt thereof, either alone, or in combination with one or more additional therapeutic agent, to a subject in need thereof. In some embodiments, the lung condition is characterized by a level of one or more inflammatory biomarkers (e.g., one or more cytokines, such as one or more of IL-6, IL-10, TNF-α, CCL2, CCL3, and CCL4) that are elevated in the subject (e.g., in lung tissue in the subject) compared to a subject that does not have the lung condition. Thus, in some embodiments, the subject is a subject that exhibits an elevated level of one or more cytokines, e.g., IL-6, IL-10, TNF-α, CCL2, CCL3, and/or CCL4 compared to a healthy subject. In some embodiments, the elevated level of one of more cytokines is also referred to herein as a “cytokine storm.” in some embodiments, the lung condition is selected from the group including, but not limited to, COVID-19 and other respiratory diseases caused by infections (e.g., viral infections such as, but not limited to, Middle East respiratory syndrome (MERS), influenza A or other influenza virus infections, respiratory syncytial virus (RSV), etc), bronchiopulmonary dysplasia, pulmonary fibrosis, and chemical exposure. Bronchiopulmonary dysplasia is a disease of premature babies after long periods on a respirator. Chemical exposure that can result in lung conditions associated with an elevated level of one or more cytokines includes, but is not limited to, exposure to chlorine, exposure to poison gases that cause lung fibrosis, and the like.

In some embodiments, the subject is a mammalian subject. In some embodiments, the subject is a non-human mammalian subject. In some embodiments, the subject is a human subject. In some embodiments, the subject is not a subject who has been diagnosed with or who is being treated for thyroid cancer. In some embodiments, the subject is not a subject who has been diagnosed with cancer or is being treated with vandetanib for cancer (e.g., thyroid cancer) or another proliferative disorder (e.g., psoriasis). In some embodiments, the subject is not a subject who has been prescribed vandetanib for treatment of another disease, disorder or condition (i.e., a disease, disorder or condition other than the lung condition associated with an elevated level of one or more cytokines).

Any suitable route of administration of the vandetanib can be used. In some embodiments, the vandetanib is administered orally. In some embodiments, the method further comprises administering to the subject one or more additional therapeutic agents, e.g., another therapeutic agent for treatment of the lung condition or a symptom thereof. In some embodiments, the one or more additional therapeutic agents include one or more antiviral (e.g., remdesivir, nirmatrelvir, ritonavir, molnupiravir) or other agent known to treat or prevent COVID-19. In some embodiments, the one or more additional therapeutic agents include one or more compounds that modify the metabolism or transport of vandetanib. For example, the one or more additional therapeutic agents can include one or more compounds that inhibit cytochrome P450 3A4 (CYP3A4), such as but not limited to, erythromycin, clarithromycin, ketoconazole, diltiazem, colchicine, a fluoroquinolone, ritonavir, verapamil, and amiodarone; and/or one or more compounds that inhibit flavin containing monooxygenase 1 (FMO1) or flavin containing monooxygenase 3 (FMO3), such as, but not limited to imipramine and (E)-3-[2-(4-(dimethylamino)phenyl)vinyl]benzoic acid. In some embodiments, the one or more additional therapeutic agents include an anti-inflammatory agent, such as, but not limited to, a corticosteroid (e.g., prednisone, dexamethasone, methylprednisone, etc,) and/or an immunosuppressant (e.g., methotrexate, cyclophosphamide, tocilzumab, etc.). Additional therapeutic agents can also include diuretics and bronchodilators, such as, but not limited to, beta-2 agonists, such as salbutamol and salmeterol, and anticholinergics, such as ipratropium. In some embodiments, the one or more additional therapeutic agents include another kinase inhibitor and/or an active metabolite of vandetanib. The one or more additional agents can be administered at about the same time (e.g., within the same 24 hour, 12 hour, or 6 hour time period) as the vandetanib or pharmaceutically acceptable salt thereof, prior to (e.g., one or more days prior to) the vandetanib or pharmaceutically acceptable salt thereof, or after (e.g., one or more days after) the vandetanib or pharmaceutically acceptable salt thereof.

In some embodiments, administration of the vandetanib or pharmaceutical salt thereof can reduce the level of one or more cytokine in the subject (e.g., in the lung tissue of the subject, as compared to an untreated subject having the lung condition). In some embodiments, the one or more reduced cytokine is an interleukin, a chemokine, or a tumor necrosis factor. In some embodiments, the one or more reduced cytokine includes one or more of interleukin-6 (IL-6), interleukin-10 (IL-10), and tumor necrosis factor alpha (TNF-α). In some embodiments, the administration reduces a level of one or more chemokine (C—C motif) ligand (CCL) in the subject. In some embodiments, the CCL is selected from the group including, but not limited to, CCL2, CCL3, and CCL4.

In some embodiments, the administration can provide an increase in a level of interferon 1β (IFN-1β) in the subject (e.g., as compared to an untreated subject having the lung condition).

In some embodiments, the presently disclosed subject matter provides a method of treating COVID-19 in a subject in need thereof. In some embodiments, the method comprises administering to the subject a therapeutically effective amount of vandetanib or a pharmaceutically acceptable salt thereof. In some embodiments the subject in need of treatment can be a subject with COVID-19 characterized by a level of one or more cytokines that are elevated in the subject compared to a subject that does not have COVID-19. In some embodiments, the subject is a mammalian subject. In some embodiments, the subject is a human subject. In some embodiments, the subject is not a subject who has been diagnosed with or who is being treated for thyroid cancer. In some embodiments, the subject is not a subject who has a cancer or is being treated for cancer or another proliferative disorder. In some embodiments, the subject is not a subject who has been prescribed or is being treated with vandetanib for treatment of another disease, disorder or condition (i.e., a disease, disorder, or condition other than COVID-19).

In some embodiments, the subject is a subject who has increased risk for severe COVID-19 (e.g., increased risk for disease requiring hospitalization, ventilation or oxygen support, ARDS, MODS and/or that results in death). Subjects with increased risk of severe disease include human subjects over the age of 45 or over the age of 65. Underlying medical conditions or comorbidities that can result in higher risk of severe COVID-19 include, but are not limited to, cancer, chronic kidney disease, chronic lung disease (e.g., chronic obstructive pulmonary disease (COPD), asthma, cystic fibrosis, pulmonary hypertension, and interstitial lung disease), dementia, diabetes. Down's syndrome, heart disease (e.g., heart failure, coronary artery disease, hypertension, etc.). HIV, a compromised or weakened immune system, liver disease (e.g., cirrhosis), being overweight (i.e., having a body mass index (BMI) >25 kg/m²) or obese (i.e., having a BMI≥30 kg/m²), pregnancy, sickle cell disease or thalassemia, a history of smoking, stroke, and having a history of substance abuse. In some embodiments, the subject is a subject who has been hospitalized for treatment of COVID-19, who has reduced blood oxygen levels, and/or who is receiving supplemental oxygen.

In some embodiments, administration of vandetanib or a pharmaceutical salt thereof can reduce the level of one or more cytokine in the subject (e.g., in lung tissue in the subject, as compared to an untreated subject who has COVID-19). In some embodiments, the one or more reduced cytokine is an interleukin, a chemokine, or a tumor necrosis factor. In some embodiments, the one or more reduced cytokine includes one or more of interleukin-6 (IL-6), interleukin-10 (IL-10), and tumor necrosis factor alpha (INF-α). In some embodiments, the administration reduces a level of one or more chemokine (C—C motif) ligand (CCL) in the subject. In some embodiments, the CCL is selected from the group including, but not limited to, CCL2, CCL3, and CCL4.

In some embodiments, the administration can provide an increase in a level of interferon 1β (IFN-1β) in the subject (e.g., as compared to an untreated subject with COVID-19).

Any suitable route of administration of the vandetanib can be used. In some embodiments, the vandetanib is administered orally.

In some embodiments, the method further comprises administering to the subject one or more additional therapeutic agents, e.g., another therapeutic agent for treatment of COVID-19. In some embodiments, the one or more additional therapeutic agents include an anti-inflammatory agent, such as, but not limited to, a corticosteroid (e.g., prednisone, dexamethasone, methylprednisone, hydrocortisone, etc.); an antiviral agent (e.g., remdesivir, nirmatrelvir, ritonavir, molnupiravir), baricitinib, tocilizumab, convalescent plasma, mesenchymal stem cells, or an anti-SARS-CoV-2 monoclonal antibody, such as, but not limited to, bebtelovimab, sotrovimab, bamlanivimab, etesevimab, casirivimab, imdevimab, tixagevimab, cilgavimab, and the like. In some embodiments, the one or more additional therapeutic agents include one or more compounds that modify the metabolism or transport of vandetanib. For example, the one or more additional therapeutic agents can include one or more compounds that inhibit cytochrome P450 3A4 (CYP3A4), such as but not limited to, erythromycin, clarithromycin, ketoconazole, diltiazem, colchicine, a fluoroquinolone, ritonavir, verapamil, and amiodarone; and/or one or more compounds that inhibit flavin containing monooxygenase 1 (FMO1) or flavin containing monooxygenase 3 (FMO3), such as, but not limited to imipramine and (E)-3-[2-(4-(dimethylamino)phenyl)vinyl]benzoic acid. The one or more additional agents can be administered at about the same time (e.g., within the same 24 hour, 12 hour, or 6 hour time period) as the vandetanib or pharmaceutically acceptable salt thereof, prior to (e.g., one or more days prior to) the vandetanib or pharmaceutically acceptable salt thereof, or after (e.g., one or more days after) the vandetanib or pharmaceutically acceptable salt thereof.

In some embodiments, the presently disclosed subject matter provides a composition for use in treating a lung condition in a subject in need of treatment thereof, wherein the composition comprises a therapeutically effective amount of vandetanib or a pharmaceutically acceptable salt thereof. In some embodiments, the lung condition is characterized by a level of one or more cytokines, e.g., IL-6, IL-10, TNF-β, CCL2, CCL3, and/or CCL4, that is/are elevated in the subject (e.g., in lung tissue in the subject) compared to a subject that does not have the lung condition. In some embodiments, the lung condition is selected from the group including, but not limited to, COVID-19 and other respiratory diseases caused by infections (e.g., viral infections, such as, but not limited to, Middle East respiratory syndrome (MERS), influenza A or other influenza virus infections, respiratory syncytial virus (RSV), etc), bronchiopulmonary dysplasia, pulmonary fibrosis, and chemical exposure. In some embodiments, the lung condition is selected from COVID-19, bronchiopulmonary dysplasia, pulmonary fibrosis, and chemical exposure.

In some embodiments, the composition is for administration orally. In some embodiments, the composition is for administration to a mammalian subject (e.g., a human subject). In some embodiments, the composition is for administration to a non-human mammalian subject. In some embodiments, the composition is for use in a subject who has not been diagnosed with or who is not being treated for thyroid cancer. In some embodiments, the composition is for use in a subject who has not been diagnosed with cancer or is not being treated with vandetanib for cancer (e.g., thyroid cancer) or another proliferative disorder (e.g., psoriasis). In some embodiments, the composition is for use in a subject who has not been prescribed vandetanib for treatment of another disease, disorder or condition (i.e., a disease, disorder or condition other than the lung condition associated with an elevated level of one or more cytokines).

In some embodiments, the composition further comprises one or more additional therapeutic agents, such as one or more of the additional therapeutic agents described hereinabove (e.g., an antiviral or other agent known to treat or prevent COVID-19, one or more compounds that modify the metabolism or transport of vandetanib, an anti-inflammatory agent, an immunosuppressant, a diuretic, a bronchodilators, another kinase inhibitor and/or an active metabolite of vandetanib). The one or more additional agents can be administered at about the same time (e.g., within the same 24 hour, 12 hour, or 6 hour time period) as the vandetanib or pharmaceutically acceptable salt thereof, prior to (e.g., one or more days prior to) the vandetanib or pharmaceutically acceptable salt thereof, or after (e.g., one or more days after) the vandetanib or pharmaceutically acceptable salt thereof.

In some embodiments, the composition reduces the level of one or more cytokine in the subject (e.g., in the lung tissue of the subject, as compared to an untreated subject having the lung condition). In some embodiments, the one or more reduced cytokine is an interleukin, a chemokine, or a tumor necrosis factor. In some embodiments, the one or more reduced cytokine includes one or more of interleukin-6 (IL-6), interleukin-10 (IL-10), and tumor necrosis factor alpha (TNF-α). In some embodiments, the composition reduces a level of one or more chemokine (C—C motif) ligand (CCL) in the subject. In some embodiments, the CCL is selected from the group including, but not limited to, CCL2, CCL3, and CCL4.

In some embodiments, the composition can provide an increase in a level of interferon 1β (IFN-1β) in the subject (e.g., as compared to an untreated subject having the lung condition).

In some embodiments, the presently disclosed subject matter provides a composition for use in treating COVID-19 in a subject in need of treatment thereof, the composition comprising a therapeutically effective amount of vandetanib or a pharmaceutically acceptable salt thereof. In some embodiments, the COVID-19 is characterized by a level of one or more cytokines that are elevated in the subject in need of treatment thereof as compared to a subject that does not have COVID-19. In some embodiments, the composition is for administration orally. In some embodiments, the composition further comprises one or more additional therapeutic agents for treating COVID-19, such as described hereinabove.

In some embodiments, the subject is a mammalian subject. In some embodiments, the subject is a human subject. In some embodiments, the subject is not a subject who has been diagnosed with or who is being treated for thyroid cancer. In some embodiments, the subject is not a subject who has a cancer or is being treated for cancer or another proliferative disorder. In some embodiments, the subject is not a subject who has been prescribed or is being treated with vandetanib for treatment of another disease, disorder or condition (i.e., a disease, disorder, or condition other than COVID-19).

In some embodiments, the subject is a subject who has increased risk for severe COVID-19 (e.g., increased risk for disease requiring hospitalization, ventilation or oxygen support, ARDS, MODS and/or that results in death).

In some embodiments, the composition can reduce the level of one or more cytokine in the subject (e.g., in lung tissue in the subject, as compared to an untreated subject who has COVID-19). In some embodiments, the one or more reduced cytokine is an interleukin, a chemokine, or a tumor necrosis factor. In some embodiments, the one or more reduced cytokine includes one or more of interleukin-6 (IL-6), interleukin-10 (IL-10), and tumor necrosis factor alpha (TNF-α). In some embodiments, the administration reduces a level of one or more chemokine (C—C motif) ligand (CCL) in the subject. In some embodiments, the CCL is selected from the group including, but not limited to, CCL2, CCL3, and CCL4.

In some embodiments, the composition can provide an increase in a level of interferon 1β (IFN-1β) in the subject (e.g., as compared to an untreated subject with COVID-19).

### IV. Pharmaceutically Acceptable Salts and Compositions

As noted above, in some embodiments, the active compounds of the presently disclosed subject matter (e.g., vandetanib) can be provided as pharmaceutically acceptable salts. Such salts include, but are not limited to, pharmaceutically acceptable acid addition salts, pharmaceutically acceptable base addition salts, pharmaceutically acceptable metal salts, ammonium and alkylated ammonium salts, and combinations thereof.

Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloracetic, trifluoroacetic, propionic, benzoie, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like.

Base addition salts include but are not limited to, ethylenediamine, N-methyl-glucamine, lysine, arginine, ornithine, choline, N, N′-dibenzylethylenediamine, chloroprocaine, diethanolamine, procaine, N-benzylphenethylamine, diethylamine, piperazine, tris (hydroxymethyl)- aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine dicyclohexylamine and the like.

Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

In some embodiments, the active agents can further be provided as a solvate.

The active compounds can be used on a sample either in vitro (for example, on isolated cells or tissues) or in vivo in a subject (i.e. living organism, such as a patient). As noted hereinabove, in some embodiments, the subject or patient is a human subject, although it is to be understood that the principles of the presently disclosed subject matter indicate that the presently disclosed subject matter is effective with respect to all vertebrate species, including mammals, which are intended to be included in the terms “subject” and “patient”. Moreover, a mammal is understood to include any mammalian species for which employing the compositions and methods disclosed herein is desirable, particularly agricultural and domestic mammalian species.

As such, the methods of the presently disclosed subject matter are particularly useful in warm-blooded vertebrates. Thus, the presently disclosed subject matter concerns mammals and birds. More particularly provided are methods and compositions for mammals such as humans and non-human primates, as well as other mammals of importance due to being endangered (such as Siberian tigers), of economic importance (animals raised on farms for consumption by humans), and/or of social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos or as pets (e.g., parrots), as well as fowl, and more particularly domesticated fowl, for example, poultry, such as turkeys, chickens, ducks, geese, guinea fowl, and the like, as they are also of economic importance to humans. Thus, also provided is the treatment of livestock including, but not limited to domesticated swine (pigs and hogs), ruminants, horses, poultry, and the like.

In some embodiments, the active compounds can include more than one of the active compounds described herein or can include one or more of the active compounds and one or more additional therapeutic agents. Thus, in some embodiments, the vandetanib or pharmaceutically acceptable salt thereof can be administered along with one or more additional therapeutic agents known in the art for treating a lung condition associated with elevated cytokines (e.g. COVID-19). The active compounds and the one or more additional therapeutic agents can be provided in a single formulation or co-administered in separate formulations at about the same time or at different times (e.g., different times within the same day, week, or month).

In some embodiments, the active compound of the presently disclosed subject matter can be administered in a pharmaceutically acceptable composition where the compound can be admixed with one or more pharmaceutically acceptable carriers. The term “pharmaceutically acceptable carrier” means a non-toxic material that does not interfere with the effectiveness of the biological activity of the active ingredients. In some embodiments, the pharmaceutically acceptable composition can also contain salts, buffering agents, preservatives, compatible carriers, and optionally other therapeutic agents.

Suitable routes of administration of an active compound or pharmaceutically acceptable composition thereof to a subject include, but are not limited to, intravenous, oral, buccal, topical, subcutaneous, intraperitoneal, pulmonary, intranasal, intracranial, intramuscular, intra-arterial, intramedullary, intrathecal, intraventricular, transdermal, enteral, sublingual, or rectal administration. The particular mode of administering a composition matter depends on various factors, including the distribution and abundance of cells to be treated and mechanisms for metabolism or removal of the composition from its site of administration.

An effective dose of a composition of the presently disclosed subject matter is administered to a subject. An “effective amount” is an amount of the composition sufficient to produce detectable treatment. Actual dosage levels of constituents of the compositions of the presently disclosed subject matter can be varied so as to administer an amount of the composition that is effective to achieve the desired effect for a particular subject and/or target. The selected dosage level can depend upon the activity of the composition and the route of administration. In some embodiments, the active compounds can be used in dosages from 0.001-1000 mg/kg body weight.

After review of the disclosure herein of the presently disclosed subject matter, one of ordinary skill in the art can tailor the dosages to an individual subject, taking into account the particular formulation, method of administration to be used with the composition, and nature of the target to be treated. Such adjustments or variations, as well as evaluation of when and how to make such adjustments or variations, are well known to those of ordinary skill in the art. In some embodiments, the vandetanib can be administered at a dose of up to about 1200 mg/day in an adult human. Thus, in some embodiments, the dose of vandetanib is about 25 mg/day to about 1200 mg/day (e.g., about 25 mg/day, 50 mg/day, 75 mg/day, 100 mg/day, 150 mg/day, 200 mg/day, 250 mg/day, 300 mg/day, 400 mg/day, 500 mg/day, 600 mg/day, 700 mg/day, 800 mg/day, 900 mg/day, 1000 mg/day, 1100 mg/day, or about 1200 mg/day). In some embodiments, vandetanib can be administered at a dose of about 100 mg/day to about 800 mg/day. In some embodiments, an adult human can be administered vandetanib at a dose of about 300 mg/day.

The therapeutically effective amount of an active agent used in a method as described herein can be determined by testing the compounds in an in vitro or in vivo model and then extrapolating therefrom for dosages in subjects of interest, e.g., humans. The therapeutically effective amount should be enough to exert a therapeutically useful effect in the absence of undesirable side effects in the subject to be treated with the composition.

Pharmaceutically acceptable carriers are well known to those skilled in the art and include, but are not limited to, from about 0.01 to about 0.1M and preferably 0.05M phosphate buffer or 0.8% saline. Such pharmaceutically acceptable carriers can be aqueous or non-aqueous solutions, suspensions and emulsions. Examples of non-aqueous solvents suitable for use in the presently disclosed subject matter include, but are not limited to, propylene glycol, polyethylene glycol, vegetable oils such as olive oil, and injectable organic esters such as ethyl oleate. Aqueous carriers suitable for use in the presently disclosed subject matter include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the presently disclosed subject matter can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the presently disclosed subject matter include, but are not limited to, water (partially containing additives as above. e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the presently disclosed subject matter include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, tale, sugars, lactose, dextrin, starch, gelatin, cellulose, polyvinylpyrrolidine, low melting waxes and ion exchange resins.

Parenteral carriers suitable for use in the presently disclosed subject matter include, but are not limited tom sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the presently disclosed subject matter can be mixed as needed with disintegrants, diluents, granulating agents, lubricants, binders and the like using conventional techniques known in the art. The carriers can also be sterilized using methods that do not deleteriously react with the compounds, as is generally known in the art. The compounds disclosed herein can take such forms as suspensions, solutions or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy A. Gennaro, ed., 20th edition, Lippincott, Williams & Wilkins. Philadelphia, Pa.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylenc-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Further, formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically scaled container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compounds can further be formulated for topical administration. Suitable topical formulations include one or more compounds in the form of a liquid, lotion, cream or gel. Topical administration can be accomplished by application directly on the treatment area. For example, such application can be accomplished by rubbing the formulation (such as a lotion or gel) onto the skin of the treatment area, or by spray application of a liquid formulation onto the treatment area.

In some formulations, bioimplant materials can be coated with the compounds so as to improve interaction between cells and the implant.

Formulations of the compounds can contain minor amounts of wetting or emulsifying agents, or pH buffering agents. The formulations comprising the compound can be a liquid solution, suspension, emulsion, tablet, pill, capsule, sustained release formulation, or powder. The compounds can be formulated as a suppository, with traditional binders and carriers such as triglycerides.

Oral formulations can include standard carriers such as pharmaceutical grades of mannitol, lactose, starch, magnesium stearate, polyvinyl pyrollidone, sodium saccharine, cellulose, magnesium carbonate, etc..

In some embodiments, the pharmaceutical composition comprising the active compound or compounds of the presently disclosed subject matter can include an agent which controls release of the compound, thereby providing a timed or sustained release compound.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Materials and Methods

Entrectinib and vandetanib were purchased from MedChemExpress (MCE, Monmouth Junction, New Jersey, United States of America).

### Pseudovirus Assay

HUVEC single cell donor (cat #C2517A, Lonza Group AG, Basel, Switzerland) cells were transduced at room temperature with ACE2 using a BacMam viral vector at a concentration of 2e⁹ VG/ml (#C11200 Pseudo SARS-CoV-2 D614G Green Reporter. Montana Molecular, Bozeman, Montana, United States of America) followed by incubation at 36° C. for 24 hours. After this step, inhibitor compounds were diluted to 1 μM and incubated for 60 minutes with 2e⁹ VG/ml of Pseudo SARS-CoV2 or Pseudo SARS-CoV2 D614G baculovirus (#C1110G or #C1120G, Montana Molecular, Bozeman, Montana, United States of America). Prior to fixation with paraformaldehyde (PFA), cell nuclei were stained with Hoechst, and images were acquired with the high content screening InCell Analyzer HS6500 microscope (20×Magnification). Quantitative analysis was done with ThermoFisher HCS Studio Cell analysis suite (ThermoFisher Scientific, Waltham, Massachusetts, United States of America.

### SARS-Cov-2 Texted in A549-ACE2 Cells

The assay was performed as previously described,¹² using A549-ACE2 mock cells or infected cells at a MOI of 0.02 with infectious clone SARS-CoV-2-nLuc⁷⁵ or mock infected with infection media to evaluate toxicity. The drugs (2×) were administered to the cells 1 hour before infection. Cytotoxicity and viral growth were evaluated 48 hours post infection with a luciferase assay sold under the tradename NANO-GLO® (Promega Corporation, Madison, Wisconsin, United States of America) and a CytoTox-Glo Cytotoxicity assay (Promega Corporation, Madison, Wisconsin, United States of America), respectively, performed per manufacturer instructions. Replication and toxicity were normalized to the vehicle wells on each plate.

### SARS-Cov-2 Tested in Calu-3 Cells

Calu-3 (ATCC, 1113-55) cells were pretreated with test compounds for 2 hours prior to continuous infection with SARS-CoV-2 (isolate USA WA1/2020) at a MOI=0.5. Forty-eight hours post-infection, cells were fixed, immunostained, and imaged by automated microscopy for infection (dsRNA+ cells/total cell number) and cell number. Sample well data was normalized to aggregated DMSO control wells and plotted versus drug concentration to determine the IC₅₀ (infection: blue when graphs viewed in color) and CC₅₀ (toxicity: green when tables viewed in color).

### SARS-Cov-2 Tested in Caco-2 Cells

The methodology for the reduction of virus yield (VYR) assay in the Caco-2 was identical to a Vero 76 cell assay previously described.¹² and the EC90 reported.

### Murine Hepatitis Virus

Each compound was tested for antiviral activity against murine hepatitis virus (MHV), a group 2a betacoronavirus, in DBT cells. MHV-A59 with nano-Luciferase: The MHV-A59 G plasmid was engineered to replace most of the coding sequence for orf4a and 4b with nano-luciferase (nLuc). Briefly, nucleotides 27,983 to 28,267 were removed and replaced with Sall and Sacll restriction sites; approximately 111 bp of the 3′ end of orf4B was left to maintain the TRS for orf5. Nano-luciferase was PCR amplified with primers 5′nLuc Sail (5′-NNNNNNGTCGACATGGTCTTCACACTCGAAGATTTC-3′; SEQ ID NO: 1) and 3′nLuc SacII (5′-NNNNNNCCGCGGTTACGCCAGAATGCGTTCCCAC-3′; SEQ ID NO: 2), digested with Sall and SacII and then cloned into the G plasmid which had been similarly digested. A sequence verified G-nLuc plasmid was used with MHV-A59 wild type A, B, C, D, F and F plasmids to recover virus expressing nLuc, using a previously described molecule clone (Systematic assembly of a full-length infectious cDNA of mouse hepatitis virus strain A59.⁷⁶ Each compound was tested against MHV using an 8-point dose response curve consisting of serial fourfold dilutions, starting at 10 μM. The same range of compound concentrations was also tested for cytotoxicity in uninfected cells.

### HCoV 229E Antiviral Assay

HCoV 229E, (a gift from Ralph Baric, University of Chapel Hill, Chapel Hill, North Carolina, United States of America) was propagated on Huh-7 cells and titers were determined by TCID₅₀, assay on Huh-7 cells. Huh-7 cells were plated at a density of 25,000 cells per well in 96 well plates and incubated for 24 h at 37° C. and 5% CO₂. Growth media was removed, and cells were pretreated with 2×drug for 1 hour prior to infection at 37C and 5% CO₂, Cells were infected with HCoV 229E at a MOI of 0.1 in a volume of 50 ul MEM 1+1+1 (Modified Eagles Medium, 1% FBS, 1% antibiotics, 1% HEPES buffer) for 1 hour. Virus was removed, cells rinsed once with PBS growth medium was added back at a volume of 100 μl. Supernatants were harvested after 24 h, serially ten-fold diluted, and virus titer was determined by TCID₅₀ assay on Huh-7 cells. CPE was monitored by visual inspection at 96 h post infection. TCID₅₀ titers were calculated using the Spearmann-Karber method.^{77,78}

### SARS-CoV-2

SARS-CoV-2 was isolated from a COVID-19 positive-tested patient. The virus was propagated and titrated in Vero E6 cells in a biosafety level 3 laboratory (BSL3) at the Ribeirao Preto Medical school (Ribeirao Preto, Brazil). Cells were cultured in DMEM medium supplemented with 10% fetal bovine serum (FBS) and antibiotic/antimycotic (Penicillin 10,000 U/mL; Streptomycin 10,000 μg/mL). The viral inoculum was added to Vero cells in DMEM (FBS 2%) incubated at 37° C. with 5% CO₂ for 48 h. The cytopathogenic effect was observed under a microscope. Cell monolayer was collected, and the supernatant was stored in −70° C. Virus titration was made by the plaque-forming units (PFU).

K18-hACE2 mice

To evaluate the effects of vandetanib in vivo, K18-hACE2 humanized mice (B6·Cg-Tg(K18-ACE2)2PrImn/J),^{42,79,80} were used. K18-hACE2 mice were obtained from The Jackson Laboratory and were breed in the Centro de Criação de Animais Especiais (Ribeirão Preto Medical School/University of São Paulo). This mouse has been used as model for SARS-CoV-2-induced disease and it presents clinical signs, and biochemical and histopathological changes compatible with the human disease.⁷⁹⁻⁸⁵ Mice had access to water and rood ad libitum. For the experimental infection, animals were transferred to the BSL3 facility.

### SARS-Co V-2 Experimental Infection and Treatments

Female K18-hACE2 mice, aged 8 weeks, were infected with 2×10⁴ PFU of SARS-CoV-2 (in 40 μL) by intranasal route. Uninfected mice were inoculated with equal volume of PBS. On the day of infection, 1 h before virus inoculation, animals were treated with vandetanib (25 mg/kg, i.p.) (n=6). Five infected animals remained untreated and used as a control group. Vandetanib was also given once daily on the days 1, 2 and 3 post-infection. Body weight was evaluated on the baseline and on all the days post-infection. On the day 3 post-infection, 6 h after treatments, animals were humanely euthanized, and lungs were collected. Right lung was collected, harvested, and homogenized in PBS with steel glass beads. The homogenate was added to TRIzol reagent (1:1), for posterior viral titration via RT-qPCR, or to lysis buffer (1:1), for ELISA assay, and stored at −70° C. The left lung was collected in paraformaldehyde (PFA 4%) for posterior histological assessment.

### Absolute Viral Copies Quantification

Total RNA from the lung was obtained using the TRIZOL® (Invitrogen, Waltham, Massachusetts, United States of America) method and quantified using a spectrophotometer sold under the tradename NANODROP One/Once (Nanodrop Technologies, Wilmington, Delaware, United States of America). A total of 800 ng of RNA was used to synthesize cDNA. cDNA was synthesized using the High-Capacity cDNA Reverse Transcription kit (Applied Biosystems, Foster City, California, United States of America), following the manufacturer's protocol. The determination of the absolute number of viral copies was made by a tagman real-time qPCR assay with the aid of a PCR system sold under the tradename STEPONE® Real-Time PCR System (Applied Biosystems, Foster City, California, United States of America). A standard curve was generated in order to obtain the exact number of copies in the tested sample. The standard curve was performed using an amplicon containing 944 bp cloned from a plasmid (PTZ57R/T CLONEJET™ Cloning Kit, Thermo Fisher Scientific, Waltham, Massachusetts, United States of America), starting in the nucleotide 14 of the gene N. To quantify the number of copies, a serial dilution of the plasmid in the proportion of 1:10 was performed. Commercial primers and probes for the N1 gene and RNAse P (endogenous control) were used for the quantification (2019-nCov CDC EUA Kit, Integrated DNA Technologies (IDT), Coralville, Iowa, United States of America), following the CDC's instructions.

### ELISA Assay

Lung homogenate was added to RIPA buffer in proportion of 1:1, and then centrifuged at 10,000 g at 4° C. for 10 minutes. Supernatant was collected and stored in −70° C. until use. The Sandwich ELISA method was performed to detect the concentration cytokines and chemokines using kits sold under the tradename DUOSET® from R&D Systems (Minneapolis, Minnesota, United States of America), according to the manufacturer. The following targets were evaluated: IL-6, IL-10, IL-1β, TNF-α, INF-1β, CCL2, CCL3, CCL4, CXCL1, CXCL2, and CXCL10.

### Lung Histopathological Process and Analyses

Five micrometer lung slices were submitted to Hematoxylin and Eosin staining. A total of 10 photomicrographs in 40×magnification per animal were randomly obtained using a microscope (Novel L3000 LED, China) coupled to a HDI camera for images capture. The total septal area and total area were analyzed with the aid of the Pro Plus 7 software (Media Cybernetics. Inc., Rockville, Maryland. United States of America). Morphometric analysis was performed in accordance with the protocol established by the American Thoracic Society and European Thoracic Society (ATS/ERS).⁸⁶

### Example 2

Vandetanib Inhibits SARS-Cov-2 Replication In Vitro without Cell Toxicity

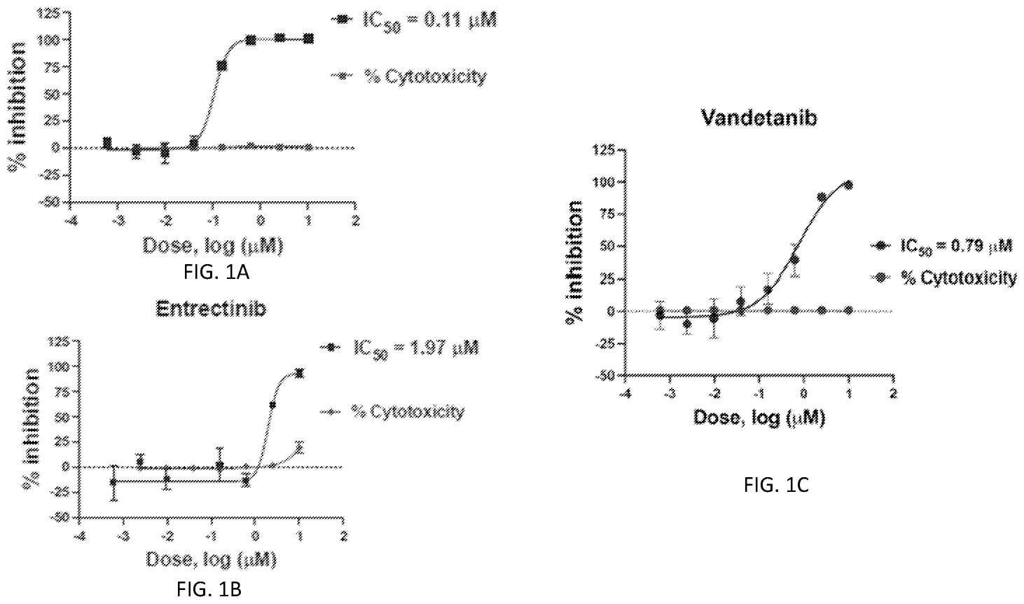

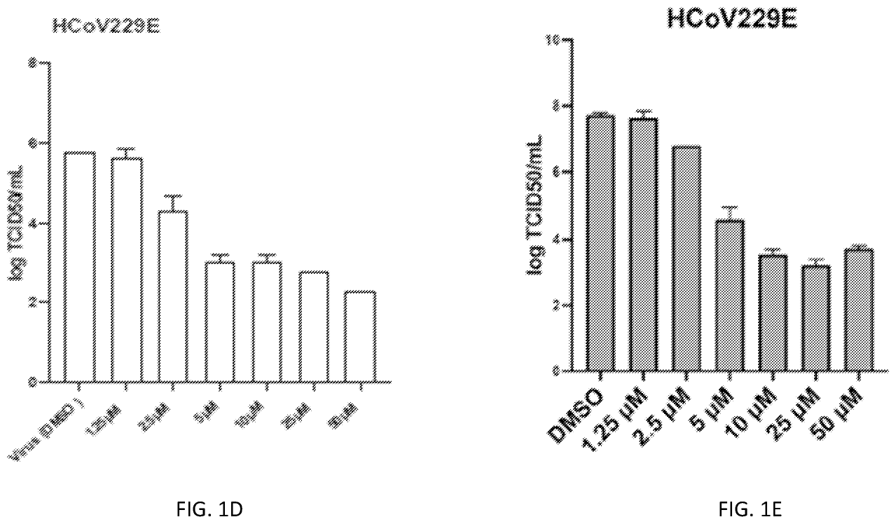

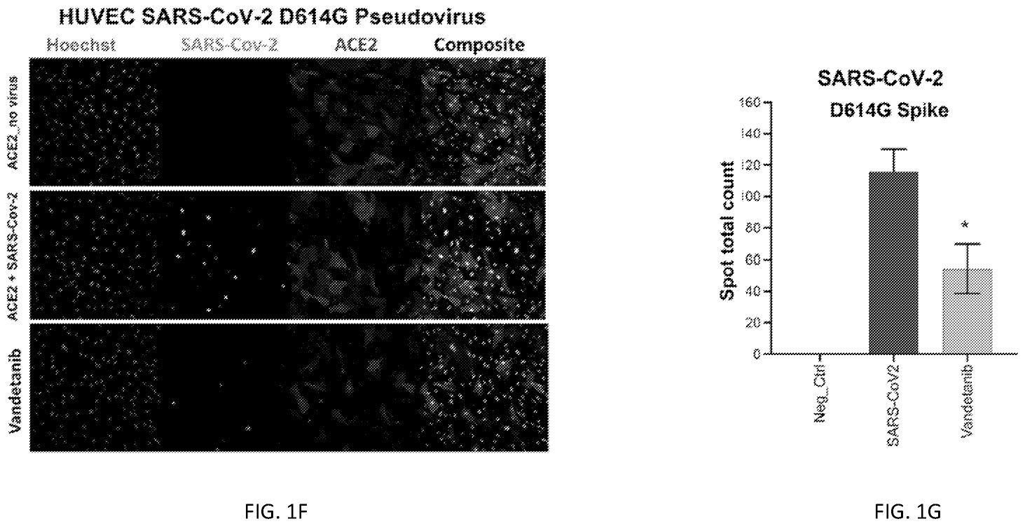

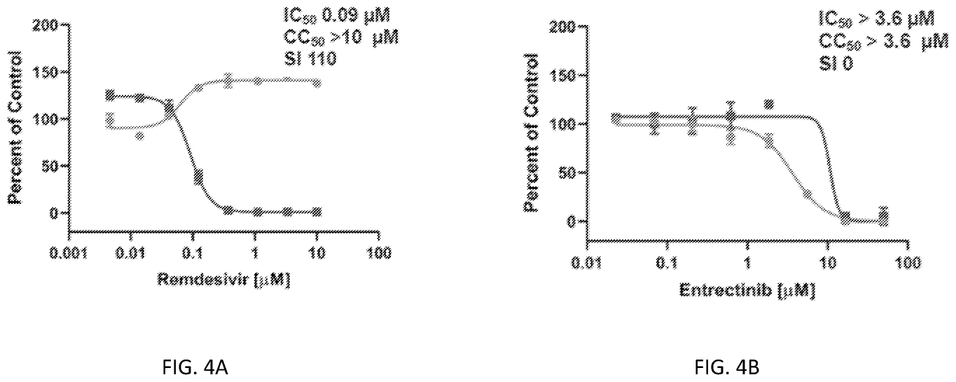

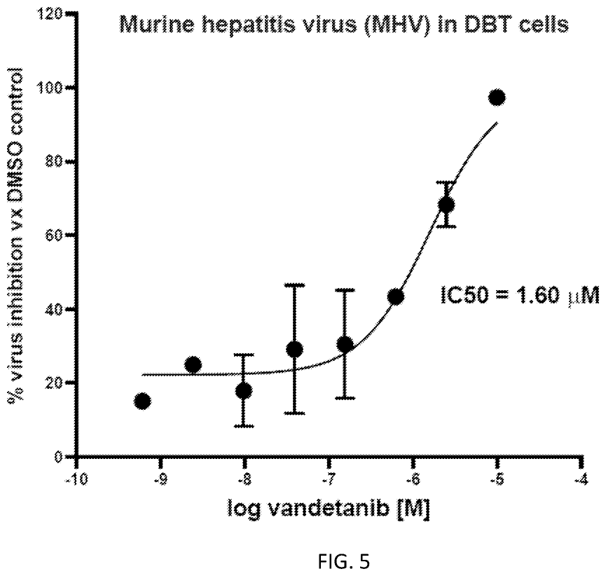

Initially, DBT cells infected with murine hepatitis virus (MI-V), a model of SARS-CoV-2 replication to evaluate the antiviral activity of 45 kinase inhibitors. See Table 1, below. Among the compounds tested, 12 showed close to 100% inhibition, and 11 showed moderate activity >60% and <83% at 10 μM. The results for vandetanib are shown in FIG. 5. Two of the most active hits from the MHV assay, entrectinib and vandetanib, were also characterized in A549-ACE2 cells infected with SARS-CoV-2 using remdesivir as a positive control. Cells were pretreated with compounds 1 h prior to SARS-CoV-2 infection. Both entrectinib (IC₅₀ 1.97 μM) and vandetanib (IC₅₀ 0.79 μM) showed lower potency than remdesivir (IC₅₀ of 0.11 μM). See FIGS. 1A-1C. Entrectinib was further tested in Caco-2 and in Calu-3 cell lines infected with SARS-CoV-2, however, this compound was toxic at the concentrations tested. See Table 2, below, and FIG. 4B. For comparison, results of remdisivir testing in Calu-3 cells are shown in FIG. 4A. Entrectinib (5 μM) was tested in Huh-7 cells infected with the human coronavirus 229E (HCoV-229E)³⁴⁻³⁶ demonstrating a decease of 2.7 logTCID50/ml. See FIG. 1D. In contrast, vandetanib was active in Caco-2 cells (IC₉₀ 2 μM)(see Table 2, below) and showed a reduction of >3 IogTCID50/mL with HCoV-229E when tested at 5 μM. See FIG. 1E. SARS-CoV-2 Spike protein-mediated entry was measured in VSV-pseudotype SARS-CoV-2 assays demonstrating that vandetanib was active at 1 μM in the SARS-CoV-2 D614G strain (see FIGS. 1F and 1G), whereas entrectinib had no significant activity.

TABLE 1
Kinase inhibitors tested against MHV and SARS-COV-2.

| | | | % Inhibition | | | | |
|---|---|---|---|---|---|---|---|
| | | | MHV-DBT | A549-ACE2 | | | |
| Compound | Structure | Target | cells 10 μM | SARS-CoV-2 | Caco-2 | Calu-3 | HCOV |
| Entrectimb | 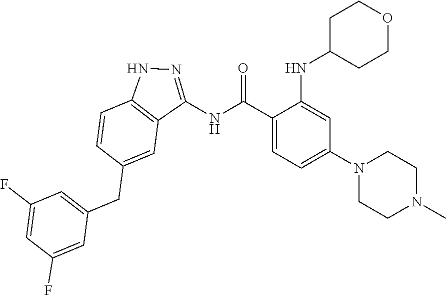 [SMILES: CN1CCN(c2ccc(C(=O)Nc3n[nH]c4ccc(Cc5cc(F)cc(F)c5)cc34)c(NC3CCOCC3)c2)CC1] | Selective tyrosine kinase inhibitor (TKI). of the tropomyosin receptor kinases (TRK) A, B and C, C-ros oncogene 1 (ROS1) and anaplastic lymphoma kinase (ALK) | 97 | 1.97 μM | N | toxic | Y |
| Vandetanib | 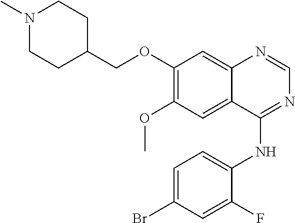 [SMILES: COc1cc2c(Nc3ccc(Br)cc3F)ncnc2cc1OCC1CCN(C)CC1] | Inhibitor of vascular endothelial growth factor receptor-2, epidermal growth factor receptor, and RET tyrosine kinases. RET tyrosine kinases | 97 | 0.79 μM | 2 μM | | Y |
| Crizotinib | 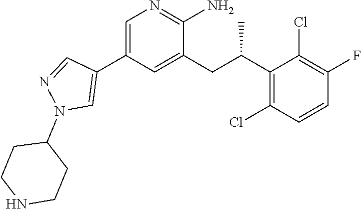 [SMILES: C[C@@H](Cc1cc(-c2cnn(C3CCNCC3)c2)cnc1N)c1c(Cl)ccc(F)c1Cl] | ALK (anaplastic lymphoma kinase) and ROS1 (c-ros oncogene 1) inhibitor | 96 | | | toxic | |
| Bosutinib | 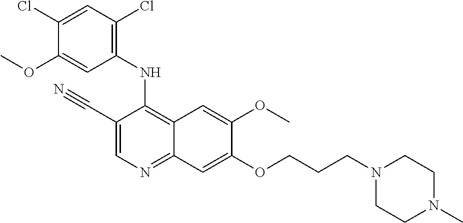 | BCR-ABL and src tyrosine kinase inhibitor | 96 | | | | |
| Axitinib | 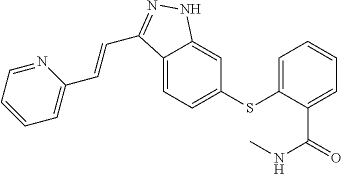 [SMILES: CNC(=O)c1ccccc1Sc1ccc2c(/C=C/c3ccccn3)n[nH]c2c1] | Its primary mechanism of action is thought to be vascular endothelial growth factor receptor 1-3, c-KIT and PDGFR inhibition | 35 | N | | | |
| Nilotinib | 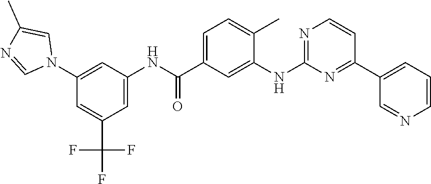 [SMILES: Cc1cn(-c2cc(NC(=O)c3ccc(C)c(Nc4nccc(-c5cccnc5)n4)c3)cc(C(F)(F)F)c2)cn1] | BCR-ABL and src tyrosine kinase inhibitor | 66 | | | | |
| Dasatinib *Clinical trials Covid-19 | 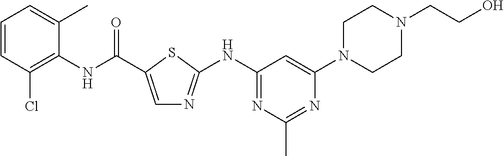 [SMILES: Cc1nc(Nc2ncc(C(=O)Nc3c(C)cccc3Cl)s2)cc(N2CCN(CCO)CC2)n1] | BCR/Abl, Src, c-Kit, ephrin receptors, and several other tyrosine kinases | 65 | Y | | | |
| Linsitinib | 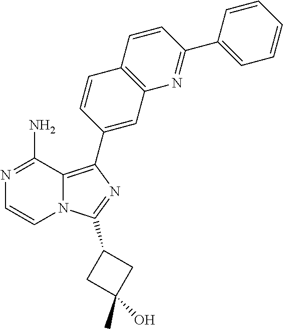 [SMILES: C[C@]1(O)C[C@@H](c2nc(-c3ccc4ccc(-c5ccccc5)nc4c3)c3c(N)nccn32)C1] | Inhibitor of the insulin receptor and of the insulin-like growth factor I receptor | 46 | | | | |
| Sorafenib | 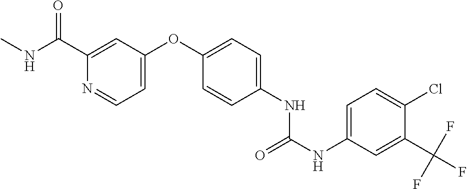 [SMILES: CNC(=O)c1cc(Oc2ccc(NC(=O)Nc3ccc(Cl)c(C(F)(F)F)c3)cc2)ccn1] | VEGFR, PDGFR and RAF kinases. | 74 | | | | |
| Pazopanib | 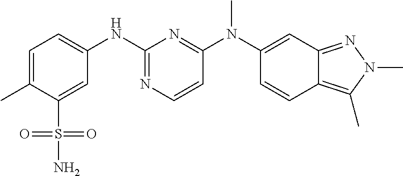 | Multi target kinase inhibitor, vascular endothelial growth factor receptor (VEGFR), platelet-derived growth factor receptor (PDGFR), c-KIT and FGFR | 20 | | | | |
| Everolimus | 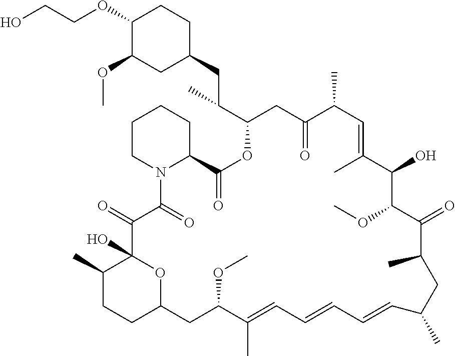 [SMILES: CO[C@H]1CC2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C] | Everolimus binds to its protein receptor FKBP12, which directly interacts with mTORC1, inhibiting its downstream signaling. | 46 | | | | |
| Midostaurin | 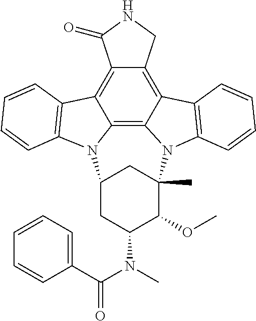 [SMILES: CO[C@@H]1[C@H](N(C)C(=O)c2ccccc2)C[C@H]2C[C@]1(C)n1c3ccccc3c3c4c(c5c6ccccc6n2c5c31)C(=O)NC4] | Active against oncogenic CD135 (FMS-like tyrosine kinase 3 receptor, FLT3) | 80 | | | | |
| Sunitinib | 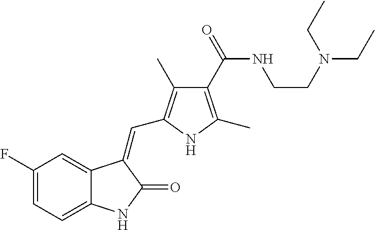 [SMILES: CCN(CC)CCNC(=O)c1c(C)[nH]c(/C=C2\C(=O)Nc3ccc(F)cc32)c1C] | Multi-targeted receptor tyrosine kinase (RTK) inhibitor. These include all receptors for platelet- derived growth factor (PDGF-Rs) and vascular endothelial growth factor receptors (VEGERs). | 96 | | | | |
| Afatinib | 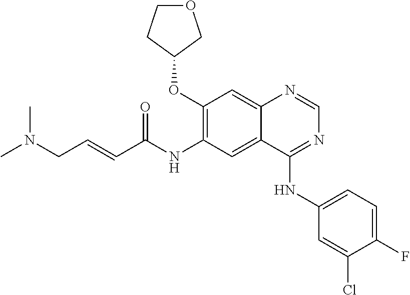 [SMILES: CN(C)C/C=C/C(=O)Nc1cc2c(Nc3ccc(F)c(Cl)c3)ncnc2cc1O[C@@H]1CCOC1] | Protein kinase inhibitor that also irreversibly inhibits human epidermal growth factor receptor 2 (Her2) and epidermal growth factor receptor (EGFR) kinases. | 98 | Y | | | |
| Regorafenib | 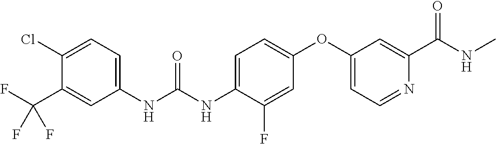 [SMILES: CNC(=O)c1cc(Oc2ccc(NC(=O)Nc3ccc(Cl)c(C(F)(F)F)c3)c(F)c2)ccn1] | Multi-kinase inhibitor, receptor tyrosine kinase (RTK. | 65 | | | | |
| Rucaparib | 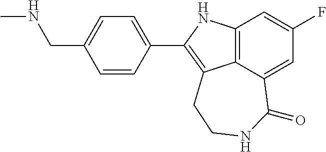 [SMILES: CNCc1ccc(-c2[nH]c3cc(F)cc4c3c2CCNC4=O)cc1] | enzyme poly ADP ribose polymerase (PARP). | −48 | | | | |
| Alisertib | 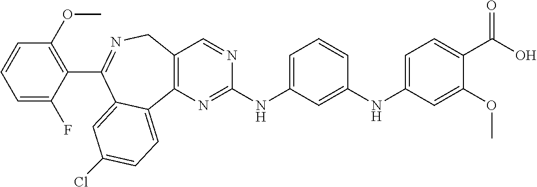 [SMILES: COc1cc(Nc2cccc(Nc3ncc4c(n3)-c3ccc(Cl)cc3C(c3c(F)cccc3OC)=NC4)c2)ccc1C(=O)O] | Aurora A kinase inhibitor | 19 | | | | |
| Tivozanib | 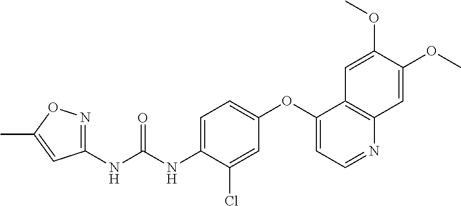 | VEGF receptor tyrosine kinase inhibitor | 29 | | | | |
| Lonvatinib | 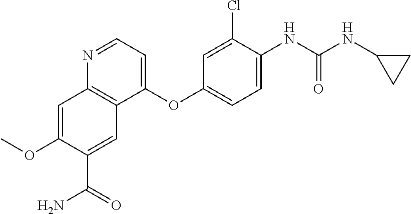 [SMILES: COc1cc2nccc(Oc3ccc(NC(=O)NC4CC4)c(Cl)c3)c2cc1C(N)=O] | Multiple kinase inhibitor against the VEGFR1, VEGFR2 and VEGFR3 kinases. | −12 | | | | |
| Ibrutinib *Clinical trials Covid-19 | 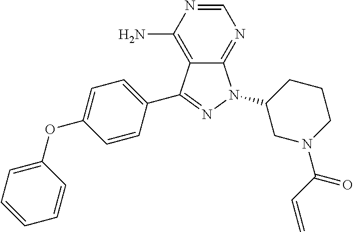 | Bruton's tyrosine kinase (BTK) | −69 | | | | |
| Trametinib | 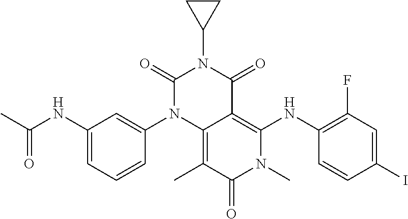 [SMILES: CC(=O)Nc1cccc(-n2c(=O)n(C3CC3)c(=O)c3c(Nc4ccc(I)cc4F)n(C)c(=O)c(C)c32)c1] | MEK inhibitor | −40 | | | | |
| Ponatinib | 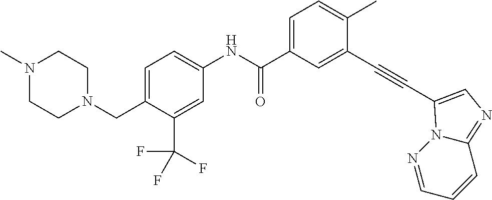 [SMILES: Cc1ccc(C(=O)Nc2ccc(CN3CCN(C)CC3)c(C(F)(F)F)c2)cc1C#Cc1cnc2cccnn12] | Multi-targeted tyrosine- kinase inhibitor, primary BCR-ABL | 98 | | | | |
| Vemourafenib | 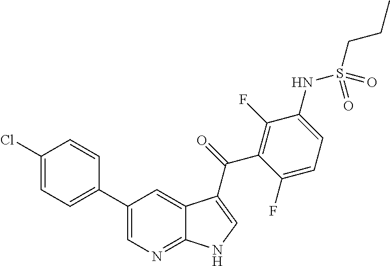 [SMILES: CCCS(=O)(=O)Nc1ccc(F)c(C(=O)c2c[nH]c3ncc(-c4ccc(Cl)cc4)cc23)c1F] | Inhibitor of the B- Raf enzyme, interrupts the B-Raf/MEK step on the B-Raf/MEK/ERK pathway-if the B-Raf has the common V600E mutation. | −3 | | | | |
| Defactinib | 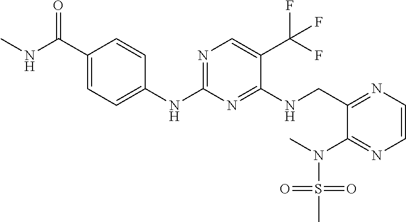 [SMILES: CNC(=O)c1ccc(Nc2ncc(C(F)(F)F)c(NCc3nccnc3N(C)S(C)(=O)=O)n2)cc1] | Inhibitor of FAK and PYK2 | 36 | | | | |
| Brigatinib | 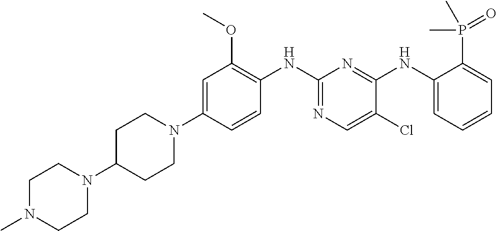 [SMILES: COc1cc(N2CCC(N3CCN(C)CC3)CC2)ccc1Nc1ncc(Cl)c(Nc2ccccc2P(C)(C)=O)n1] | Anaplastio lymphoma kinase (ALK) and epidermal growth factor receptor (EGFR) inhibitor. | 39 | N | | | |
| Alectinib | 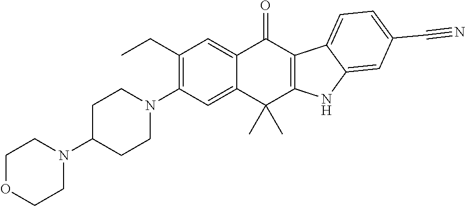 [SMILES: CCc1cc2c(cc1N1CCC(N3CCOCC3)CC1)C(C)(C)c1[nH]c3cc(C#N)ccc3c1C2=O] | Anaplastic lymphoma kinase (ALK) | 62 | | | | |
| Cabozantinib | 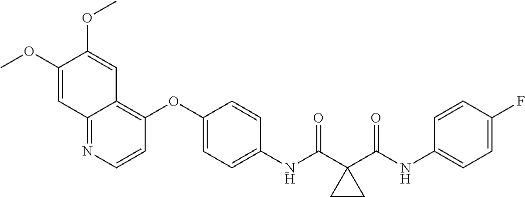 [SMILES: COc1cc2nccc(Oc3ccc(NC(=O)C4(C(=O)Nc5ccc(F)cc5)CC4)cc3)c2cc1OC] | Tyrosine kinases c- Met and VEGFR2, and also inhibits AXL and RET. | 61 | | | | |
| Idelalisib | 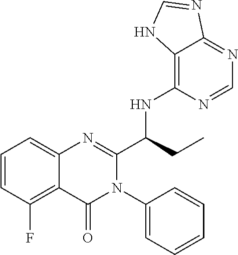 [SMILES: CC[C@H](Nc1ncnc2nc[nH]c12)c1nc2cccc(F)c2c(=O)n1-c1ccccc1] | Phosphoinositide 3- kinase inhibitor | 17 | | | | |
| Cobimetinib | 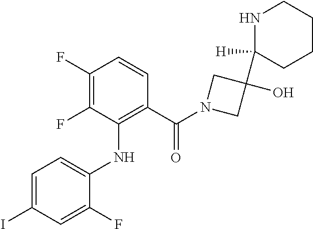 [SMILES: O=C(c1ccc(F)c(F)c1Nc1ccc(I)cc1F)N1CC(O)([C@@H]2CCCCN2)C1] | MEK inhibitor | 82 | | | | |
| Galunisertib | 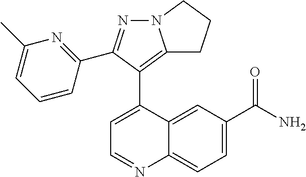 [SMILES: Cc1cccc(-c2nn3c(c2-c2ccnc4ccc(C(N)=O)cc24)CCC3)n1] | TGF-β receptor type 1 kinase inhibitor | 26 | | | | |
| Flumatinib | 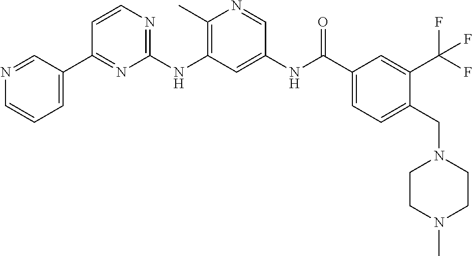 [SMILES: Cc1ncc(NC(=O)c2ccc(CN3CCN(C)CC3)c(C(F)(F)F)c2)cc1Nc1nccc(-c2cccnc2)n1] | Inhibits the wild-type forms of Bcr-Abl. platelet-derived growth factor receptor (PDGFR) and mast/stem cell growth factor receptor (SCFR; c-Kit) | 83 | | | | |
| Dabrafenib | 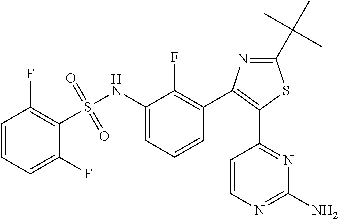 [SMILES: CC(C)(C)c1nc(-c2cccc(NS(=O)(=O)c3c(F)cccc3F)c2F)c(-c2ccnc(N)n2)s1] | Inhibitor of the associated enzyme B-Raf | −12 | | | | |
| Imatinib *Clinical trials Covid-19 | 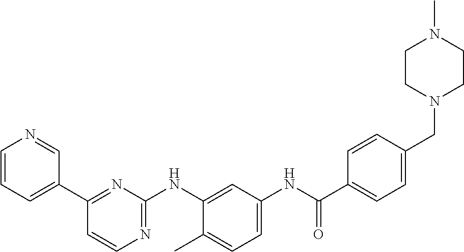 [SMILES: Cc1ccc(NC(=O)c2ccc(CN3CCN(C)CC3)cc2)cc1Nc1nccc(-c2cccnc2)n1] | Imatinib is specific for the TK domain in abl (the Abelson proto-oncogene), c- kit and PDGF-R (platelet-derived growth factor receptor). | 81 | | | | |
| Ceritinib | 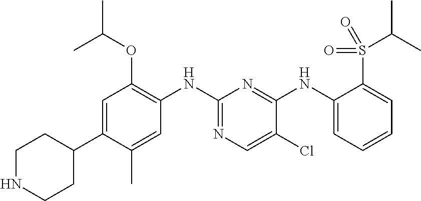 [SMILES: Cc1cc(Nc2ncc(Cl)c(Nc3ccccc3S(=O)(=O)C(C)C)n2)c(OC(C)C)cc1C1CCNCC1] | Potent inhibitor of anaplastic lymphoma kinasc (ALK) | 94 | | | | |
| Osimertinib | 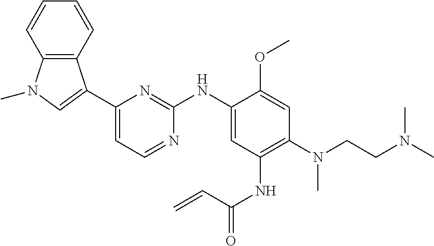 | Third-generation epidermal growth factor receptor tyrosine kinase inhibitor. | 98 | | | | |
| Ribociclib | 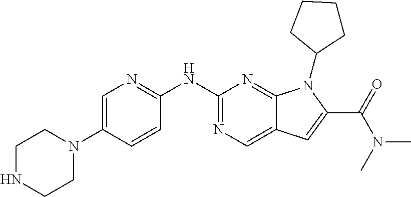 [SMILES: CN(C)C(=O)c1cc2cnc(Nc3ccc(N4CCNCC4)cn3)nc2n1C1CCCC1] | Inhibitor of cyclin D1/CDK4 and CDK6 | −4 | | | | |
| Cerdulatinib | 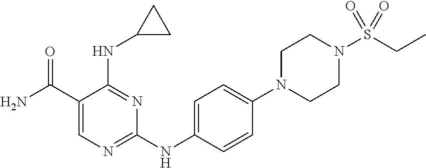 [SMILES: CCS(=O)(=O)N1CCN(c2ccc(Nc3ncc(C(N)=O)c(NC4CC4)n3)cc2)CC1] | SYK/JAK kinase inhibi tor in development for treatment ofhematological malignancies, It has lowest nM IC₅₀ values against TYK2, JAK1, JAK2, JAK3, FMS, and SYK | 78 | N | | | |
| Tofacitinib *Clinical trials Covid-19 | 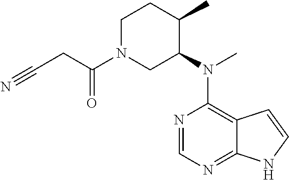 [SMILES: C[C@@H]1CCN(C(=O)CC#N)C[C@@H]1N(C)c1ncnc2[nH]ccc12] | Inhibitor of the enzyme janus kinase 1 (JAK1) and janus kinase 3 (JAK 3) | −7 | | | | |
| Palbociclib | 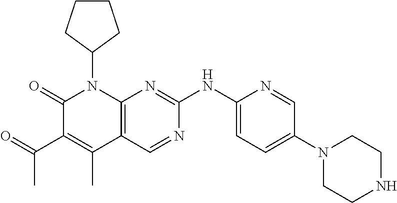 [SMILES: CC(=O)c1c(C)c2cnc(Nc3ccc(N4CCNCC4)cn3)nc2n(C2CCCC2)c1=O] | Selective inhibitor of the cyclin-dependent kinases CDK4 and CDK6 | 5 | | | | |
| Ruxolitinib *Clinical trials Covid-19 | 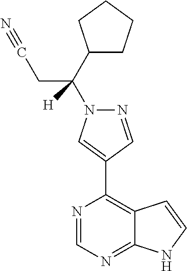 [SMILES: N#CC[C@H](C1CCCC1)n1cc(-c2ncnc3[nH]ccc23)cn1] | Janus kinase inhibitor (JAK inhibitor) with selectivity for subtypes JAK1 and JAK2 | −46 | | | | |
| Gefitinib | 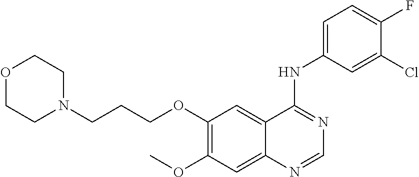 [SMILES: COc1cc2ncnc(Nc3ccc(F)c(Cl)c3)c2cc1OCCCN1CCOCC1] | Inhibitor of epidermal growth factor receptor's (EGFR) tyrosine kinase domain | 81 | Y | | | |
| Erlotinib | 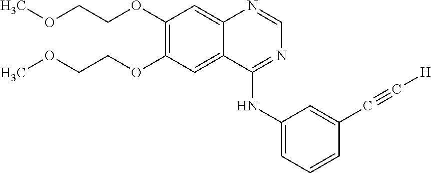 [SMILES: C#Cc1cccc(Nc2ncnc3cc(OCCOC)c(OCCOC)cc23)c1] | Inhibitor epidermal growth factor receptor (BGFR) | 32 | | | | |
| Lapatinib | 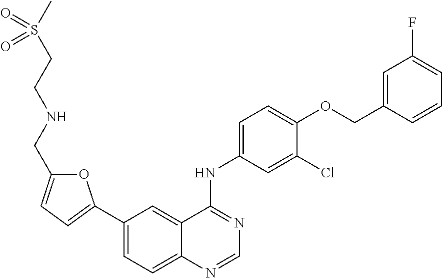 [SMILES: CS(=O)(=O)CCNCc1ccc(-c2ccc3ncnc(Nc4ccc(OCc5cccc(F)c5)c(Cl)c4)c3c2)o1] | It is a dual tyrosine kinase inhibitor which interrupts the HER2/neu and epidermal growth factor receptor (EGFR) pathways | 88 | | | | |
| Nintedanib *Clinical trials Covid-19 | 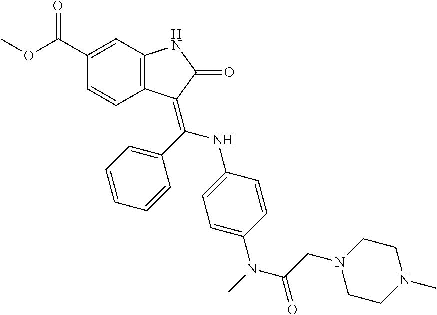 | Competitively inhibits both nonreceptor tyrosine kinases (nRTKs) and receptor tyrosine kinases (RTKS). NRTK targets of nintedanib include Lck, Lyn, and Src. RTK targets of Bintedanib include platelet-derived growth factor receptor (PDGFR) and β; fibroblast growth factor receptor (FGFR) 1, 2, and 3; vascular endothelial growth factor receptor (VEGFR) 1, 2, and 3; and FLT3. | 95 | | | | |
| Temsirolimus | 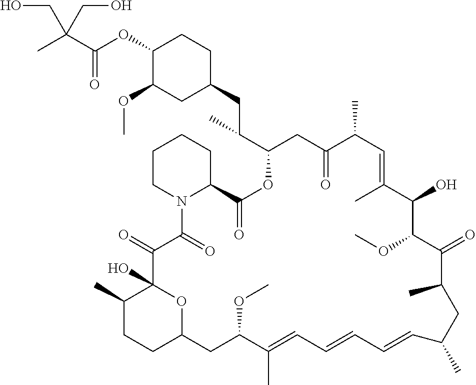 [SMILES: CO[C@H]1CC2CC[C@@H](C)[C@@](O)(O2)C(=O)C(=O)N2CCCC[C@H]2C(=O)O[C@H]([C@H](C)C[C@@H]2CC[C@@H](OC(=O)C(C)(CO)CO)[C@H](OC)C2)CC(=O)[C@H](C)/C=C(\C)[C@@H](O)[C@@H](OC)C(=O)[C@H](C)C[C@H](C)/C=C/C=C/C=C/1C] | Inhibitor of mTOR | 4 | | | | |

TABLE 2
EC₉₀, CC₅₀, and SI values for vandetanib and entrectinib tested
against SARS-CoV-2 (strain USA_WA1/2020) in Caco-2 cells.

| Compound | EC₉₀ (μM) | CC50 (μM) | SI |
|---|---|---|---|
| Entrectinib | >3.6 | 3.6 | 0 |
| Vandetanib | 2 | 4.1 | 2.1 |

### Example 3

### In Vivo Efficacy of Vandetanib in Mice

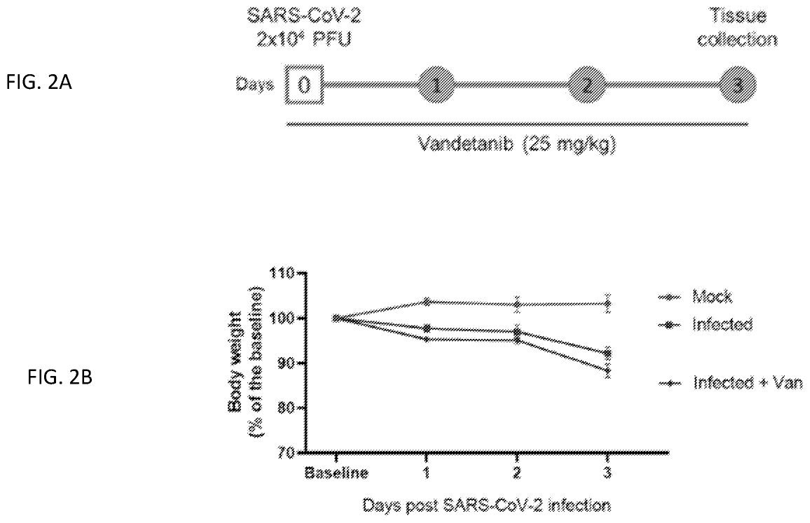

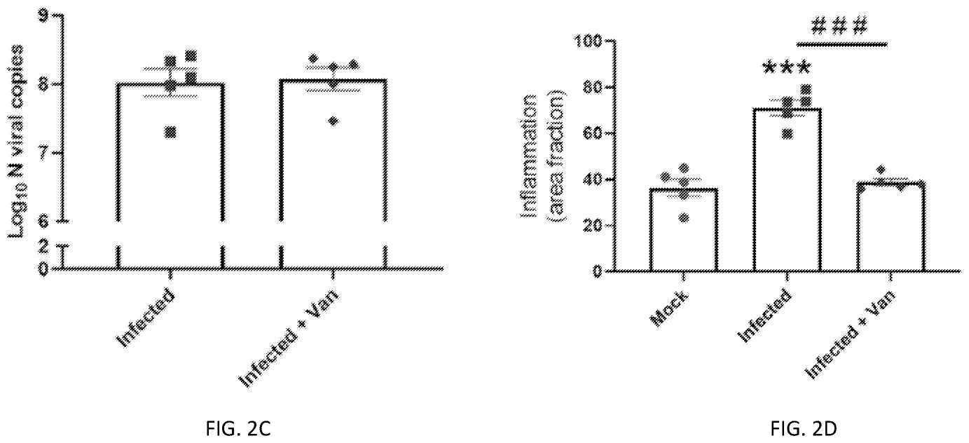

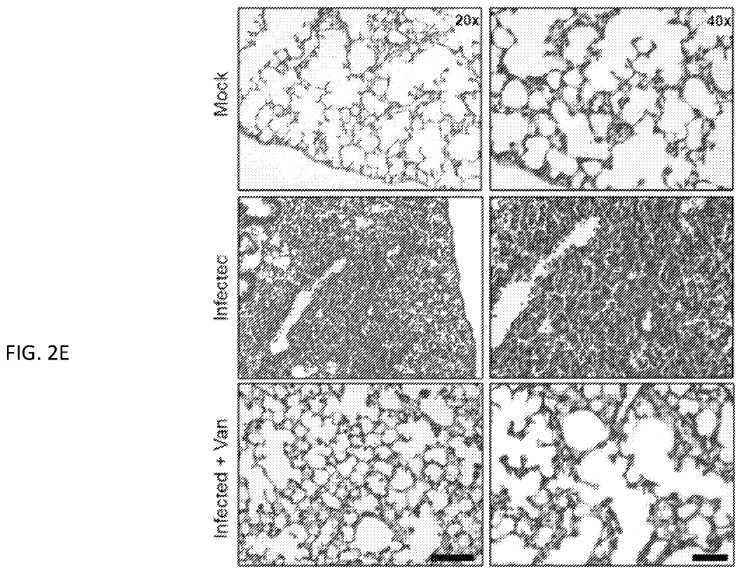

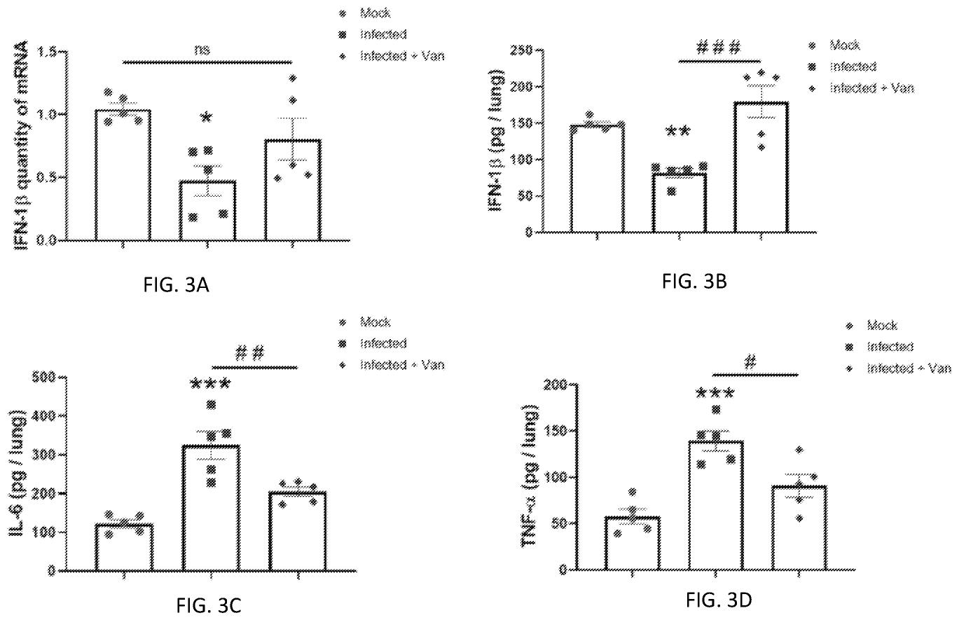

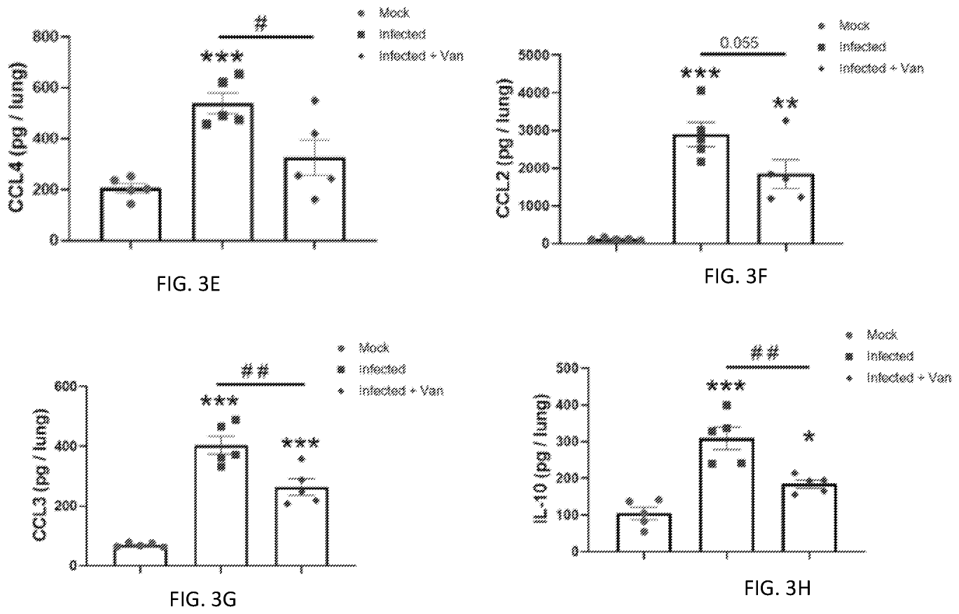

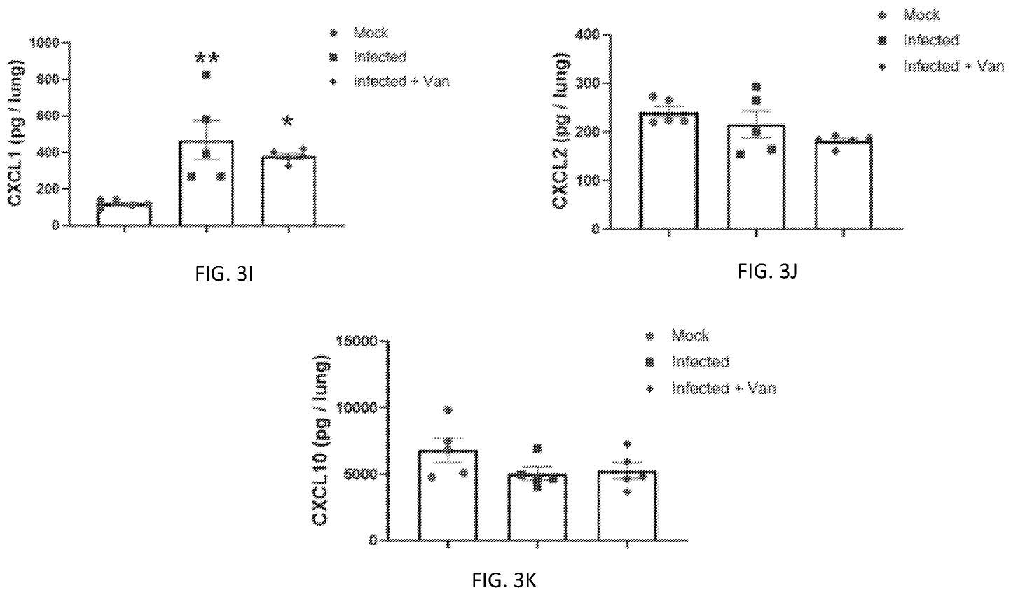

Vandetanib in vivo efficacy was assessed in the K-18-hACE2 mouse model of COVID-19⁴²⁻⁴⁴ (8-week-old females, challenged with SARS-Cov-2 2×10⁴ PFU, intranasally). Vandetanib (25 mg/kg) was administered i.p. 1 h before infection and once daily up to day 3 post-infection (3 dpi). See FIG. 2A. On 3 dpi, mice were euthanized, and viral load, cytokines and lung histopathology were evaluated. In all groups tested, mice lost weight compared to uninfected animals that received only vehicle formulation. See FIG. 2B. Lung viral load was evaluated by RT-PCR, and, although vandetanib reduced SARS-Cov-2 infection in A549-ACE2 cells, no significant reduction in viral RNA levels was observed in vivo when compared with the infected untreated mice. See FIG. 2C. However, while vandetanib did not decrease the viral load, it had a clear protective effect on lung pathology. See FIGS. 2D and 2E. Infected untreated mice showed severe pathological changes with inflammatory cell infiltrates. In contrast, vandetanib treatment exhibited improved morphology and milder infiltration even in the absence of effect on viral load. These results indicate an effect on the virus-induced inflammatory process. Further analyses also showed that vandetanib treatment restored the levels of IFN-1β (see FIGS. 3A and 3B) and prevented the increase of the levels of the most widely evaluated inflammatory cytokines/chemokines observed in infected mice. Vandetanib reduced IL-6, TNF-α, and CCL4 (compared to infected untreated animals) to similar levels found in uninfected animals. See FIGS. 3C-3E. Vandetanib also significantly reduced the levels of CCL2, CCL3, and IL-10 compared to infected animals. See FIGS. 3F-3H. CXCL1 was not affected by the treatment. See FIG. 3I. CXCL2 and CXCL10 were not elevated in infected animals. See FIGS. 3J and 3K). Altogether, and without being bound to any one theory, these results indicate that the in vivo efficacy of vandetanib in the COVID-19 mouse model is more likely related to the reduction in the cytokine storm than reduction in SARS-CoV-2 replication.

### Discussion of Examples

With a mounting infection and death toll to date, there has been a focus on ensuring the global population are vaccinated against SARS-CoV-2. Still, rapidly emerging viral variants can hamper the effectiveness of vaccines in the future,⁴⁵ while many countries still have little or no access to the vaccines that are available in the USA and Europe.⁴⁶ There is also a need to develop treatments that do not require cold chain storage and can be used outside of hospitals. Repurposing approved small molecule drugs represents a rapid route to the clinic if they can demonstrate statistically significant efficacy in animal models.

Growth factor receptor signaling pathways have been reported to be highly activated upon SARS-CoV-2 infection, hence inhibition of these pathways prevents replication in cells.⁴⁷ Vandetanib is an FDA approved drug used to treat thyroid gland tumors (targeting VEGFR, EGFR and RET-tyrosine kinase⁴⁸) and was active in Caco-2 cell, A549-ACE2 infected by SARS-CoV-2 and against HCoV-229E. Based on this in vitro activity profile, it was selected for testing in a COVID-19 mouse model.

One of the major causes of ARDS and multi-organ dysfunction syndrome (MODS) observed in severe SARS-CoV-2 infection is the cytokine storm.¹⁷⁻⁴⁹ Studies have revealed higher levels of cytokine storm associated with more severe COVID development.¹⁷ In these patients, accumulation and exudation of inflammatory substances from the cytokine storm destroys tissues, leading to multi-organ failure and ARDS,⁴⁹ a notable cause of death in severe and critical cases of COVID-19. Capillary leakage is also major component of deteriorating lung function in COVID-19, resulting in ARDS due to inflammation driven by TNF, IL-1, IL-6, IL-8, and VEGF.¹⁷ Abnormal coagulation can result in organ failure and death in patients with severe COVID-19, caused by the cytokine storm.¹⁷ IL-1, IL-6, TNF, signal transducer and transcriptional activator 3 (STAT3), NF-κB, and lipopolysaccharides were all identified as regulators of thrombotic markers in COVID-19 patients with the severe-to-critical disease.⁵⁰

As described herein, evaluation of the in vivo efficacy of vandetanib in a murine model of infection demonstrated that vandetanib reduces IL-6, IL-10, and TNF-α compared to infected untreated animals to similar levels found in uninfected animals. In patients with severe COVID-19, the levels of several inflammatory cytokines and chemokines including PDGF, TNF, IL-6, and VEGF are significantly increased.⁵¹ IL-6 has also been shown to correlate with respiratory failure and adverse clinical outcomes.⁴⁹⁻⁵² Furthermore, a recent clinical study showed that the ARDS group had higher levels of IL-6, IL-8, and IL-10 than the non-ARDS group, and the levels of these cytokines correlated significantly with coagulation parameters and disseminated intravascular coagulation (DIC).⁴⁹ The levels of IL-6 and TNF-α correlated with the levels of creatinine and urea nitrogen and were also higher in ARDS patients with acute kidney injury (AKIU).⁴⁹ Thus, reducing the levels of these cytokines upon treatment with vandetanib can improve prognosis in COVID-19. The host innate immune response releases cytokines such as type I interferon (IFN α and β) during infection, thereby initiating antiviral activity. However, this particular interferon response is interrupted by factors such as SARS-CoV-2 non-structural proteins, aging, diabetes, and germ-line errors eventually making the host more susceptible to illness.⁵³⁻⁵⁷ One interesting observation provided herein is that vandetanib rescued the decreased IFN-β caused by SARS-CoV-2 infection in mice to levels similar to that in uninfected animals. See FIG. 3A. Vandetanib also decreased CCL2, CCL3, CCL4 and CXCL1 compared to infected animals. See FIG. 3F-3J. These chemokines have been reported to be increased in patients with COVID-19.¹⁹ CXCL1 is a known chemoattractant for neutrophils, and it was identified, among others, to be the one of the more abundantly expressed by macrophages involved in SARS-CoV2 infection, especially in more severe cases:⁵⁸ CCL2 is one of the key chemokines that regulate migration and infiltration of monocytes/macrophages and CCL2 levels were also independently associated (together with other immune soluble biomarkers) with mortality in COVID-19 patients.⁵⁹ Finally, RNA isolated from nasopharyngeal swab samples from COVID-19 positive patients showed that CCL2 (and CCL3) expression was significantly higher in patients with unfavorable outcomes than cases with a favorable evolution or with negative controls.⁶⁰ Therefore, while it is not desired to be bound by any particular theory of operation, it is believed that targeting chemokine/chemokine receptor binding can suppress hyper immune activation in critical COVID-19 patients.⁵⁸

The lung histological examination for mice treated with vandetanib showed a significant treatment effect with mild infiltration, looking similar to uninfected mice. ARDS and dyspnea create hypoxia in lung tissues and other organs. Hypoxia induces VEGF expression. VEGF is a potent vascular permeability factor that induces vascular leakiness in COVID-19 infected lung tissues, resulting in plasma extravasation and pulmonary edema, which further increases tissue hypoxia^{61,62} and VEGF participates in lung inflammation.⁶³ Blocking VEGF and the VEGF receptor (VEGFR)-mediated signaling can improve oxygen perfusion and anti-inflammatory response and alleviate clinical symptoms in patients with severe COVID-19. In agreement, bevacizumab, a humanized anti-VEGF monoclonal antibody was employed for treating patients with severe COVID-19 (NCT04275414).⁶⁴ Relative to comparable controls, bevacizumab showed clinical efficacy by improving oxygenation and shortening oxygen-support duration, and by day 28, 92% of patients show improvement in oxygen-support status, 65% patients were discharged, and none showed worsened oxygen-support status nor died. Therefore modulating VEGF using drugs such as vandetanib⁴⁸ can have clinical relevance

Many FDA-approved kinase inhibitors have been proposed as broad-spectrum antiviral therapies⁶⁵ because they have multiple protein targets, including those in the host cell required for viral life cycle, replication, and infection of multiple virus types. Host-target antivirals also offer the advantage that they can exploit the host protein and pathways needed for the replication of the virus and resistance can be less likely to develop against them.

Although it was observed in the present studies that vandetanib reduced infection in i) DBT cells infected by MHV and in ii) A549-ACE2 infected by SARS-CoV-2, iii) showed a reduction of >3 logTCID50/mL of HCoV-229E and iv) decreased viral entry in the pseudovirus assay, a statistically significant reduction in the viral load was not observed in the murine SARS-CoV-2 infected model. A decrease in viral load was also not observed in a group treated with remdesivir. Remdesivir is degraded by a serum esterase, and to perform studies in mice that mirror the pharmacokinetics and exposure seen in humans, Ceslc^{-/-} mice, which lack the serum esterase, can be used.⁶⁶ While the present results represent a suboptimal remdesivir dose it demonstrated a positive effect on the lung pathology in mice comparable to vandetanib. The positive effects shown herein regarding modulation of the main inflammatory cytokines and chemokines, as well as prevention of lung damage and fibrosis, demonstrated that vandetanib likely has the ability to address the cytokine storm associated with SARS-CoV-2 infection. Although the antiviral remdesivir has been shown to reduce the length of hospital stay for those with COVID-19,⁶⁷ to date, only anti-inflammatory approaches have improved survival in these patients, such as dexamethasone when given to those with an oxygen requirement.⁶⁸ A randomized, placebo-controlled trial of Janus-kinase inhibition using tofacitinib has been reported to improve COVID-19 survival, in the presence of background glucocorticoid treatment (received by 89% of patients).⁶⁹ The SAVE-MORE trial showed that blockade of the cytokine IL-1 via the recombinant human IL-1 receptor antagonist anakinra in patients hospitalized with moderate and severe COVID-19 reduced the risk of worse clinical outcome.⁷⁰ Now, as described herein, it has been observed that vandetanib can decrease levels of several such important cytokines that are significantly elevated in the cytokine storm, such as IL-6 and TNF-α.

In conclusion, the presently disclosed results indicate that the FDA approved kinase inhibitor vandetanib is a small molecule agent that can target the cytokine storm and prevent patients from developing ARDS. Treatment with vandetanib in the mouse model reduced key inflammatory cytokines. Vandetanib is well absorbed from the gut, reaching peak blood plasma concentrations 4 to 10 h after application, and has an average half-life of 19 days.^{72,73} The pharmacokinetic properties of vandetanib have been reported to be linear over the dosage range of 50 to 1200 mg/day. The maximum plasma concentration of 857 ng/mL is usually reached after 6 h in patients with thyroid medullary cancer at a dose of 300 mg. Vandetanib is highly protein-bound (92-94%) and has a terminal excretion half-life of 20 days. It is metabolized by cytochrome P450 3A4 (CYP3A4) and is predominantly excreted via the feces and urine (44% and 25%, respectively).⁴⁸ In another study in healthy patients with dose escalation up to 1200 mg/day, vandetanib appeared to be well tolerated in the populations studied and at the dose of 800 mg/kg, vandetanib can reach a Cₘₐₓ of 1 μM.⁷² which is above the IC₅₀ reported in the presently disclosed study of infection of SARS-CoV-2 in A549-ACE2 cells. Vandetanib is an FDA-approved drug sold under the brand name Caprelsa (Sanofi Genzyme, South San Francisco, California, United States of America) in dosage forms of 100 and 300 mg. In the presently disclosed mouse studies, a dose of 25 mg/kg was used, which can be extrapolated⁸⁷ to a daily human dose of approximately 300 mg. When combined, these pharmacokinetic effects and positive impacts on the cytokine storm and preventing lung inflammation, implicate the use of vandetanib for treating COVID-19.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicant reserves the right to challenge the accuracy and pertinence of any cited reference.
1. Rehman, M. F. U. et al. Novel coronavirus disease (COVID-19) pandemic: A recent mini review. Comput Struct Biotechnol J 19, 612-623 (2021).
2. Kyriakidis, N. C., López-Cortés, A., González, E. V., Grimaldos, A. B. & Prado, E. O. SARS-CoV-2 vaccines strategies: a comprehensive review of phase 3 candidates. NPJ Vaccines 6, 28 (2021).
3. Huang, H. Y. et al. Landscape and progress of global COVID-19 vaccine development. Hum Vaccin Immunother, 17, 3276-3280 (2021).
4. Eastman, R. T. et al. Remdesivir: A Review of Its Discovery and Development Leading to Emergency Use Authorization for Treatment of COVID-19. ACS Cent Sci 6, 672-683 (2020).
5. Kalil, A. C. et al. Baricitinib plus Remdesivir for Hospitalized Adults with Covid-19. N Engl J Med 384, 795-807 (2021).
6. Horby, P. et al. Dexamethasone in Hospitalized Patients with Covid-19. N Engl J Med 384, 693-704 (2021).
7. Apaydin, C. B., Cinar, G. & Cihan-Ustundag, G. Small-molecule antiviral agents in ongoing clinical trials for COVID-19. Curr Drug Targets 22, 1986-2005 (2021).
8. Nalbandian, A. et at. Post-acute COVID-19 syndrome. Nat Med 27, 601-615 (2021).
9. Silva Andrade, B. et at. Long-COVID and Post-COVID Health Complications: An Up-to-Date Review on Clinical Conditions and Their Possible Molecular Mechanisms. Viruses 13(4), 700 (2021).
10. Ekins, S. et al. Deja vu: Stimulating open drug discovery for SARS-CoV-2. Drug Discov Today 25, 928-941 (2020).
11. Muratov, E. N. et al. A critical overview of computational approaches employed for COVID-19 drug discovery. Chem Soc Rev 50, 9121-9151 (2021).
12. Puhl, A. C. et at. Repurposing the Ebola and Marburg Virus Inhibitors Tilorone, Quinacrine, and Pyronaridine: In Vitro Activity against SARS-CoV-2 and Potential Mechanisms. ACS Omega 6, 7454-7468 (2021).
13. Riva, L. et al. Discovery of SARS-CoV-2 antiviral drugs through large-scale compound repurposing. Nature 586, 113-119 (2020).
14. Amin, S. A., Banerjee, S., Ghosh, K., Gayen. S. & Jha, T. Protease targeted COVID-19 drug discovery and its challenges: Insight into viral main protease (Mpro) and papain-like protease (PLpro) inhibitors. Bioorg Med Chem 29, 115860 (2021).
15. Hoffman, R. L. et al. Discovery of Ketone-Based Covalent Inhibitors of Coronavirus 3CL Proteases for the Potential Therapeutic Treatment of COVID-19. J Med Chem 63, 12725-12747 (2020).
16. Costela-Ruiz, V. J., Illescas-Montes, R., Puerta-Puerta, J. M., Ruiz, C. & Melguizo-Rodriguez, L. SARS-CoV-2 infection: The role of cytokines in COVID-19 disease. Cytokine Growth Factor Rev 54, 62-75 (2020).
17. Chen, R. et al. Cytokine Storm: The Primary Determinant for the Pathophysiological Evolution of COVID-19 Deterioration. Front Immunol 12, 589095 (2021).
18. Roshanravan, N., Seif, F., Ostadrahimi, A., Pouraghaei, M. & Ghaffari, S. Targeting Cytokine Storm to Manage Patients with COVID-19: A Mini-Review. Arch Med Res 51, 608-612 (2020).
19. Coperchini, F. et al. The cytokine storm in COVID-19: Further advances in our understanding the role of specific chemokines involved. Cyokine Growth Factor Rev 58, 82-91 (2021).
20. Felsenstein, S., Herbert. J. A., McNamara, P. S. & Hedrich. C. M. COVID-19: Immunology and treatment options. Clin Immunol 215, 108448 (2020).
21. Peter, A. E., Sandeep, B. V., Rao, B. G. & Kalpana, V. L. Calming the Storm: Natural Immunosuppressants as Adjuvants to Target the Cytokine Storm in COVID-19. Front Pharmacol 11, 583777 (2020).
22. Moradian, N. et al. Cytokine release syndrome: inhibition of pro-inflammatory cytokines as a solution for reducing COVID-19 mortality. Eur Cytokine Netw 31, 81-93 (2020).
23. Beerli, C. et a). Vaccinia virus hijacks EGFR signalling to enhance virus spread through rapid and directed infected cell motility. Nat Microbiol 4, 216-225 (2019).
24. Pleschka, S. et at. Influenza virus propagation is impaired by inhibition of the Raf/MEK/ERK signalling cascade. Nat Cell Biol 3, 301-305 (2001).
25. Roskoski, R. Properties of FDA-approved small molecule protein kinase inhibitors: A 2021 update. Pharmacol Res 165, 105463 (2021).
26. Weisberg, B. et at. Repurposing of Kinase Inhibitors for Treatment of COVID-19. Pharm Res 37, 167 (2020).
27. Baranov, M. V., Bianchi, F. & van den Bogaart, G. The PIKfyve Inhibitor Apilimod: A Double-Edged Sword against COVID-19. Cells 10 (2020).
28. Hoang, T. N. et at. Baricitinib treatment resolves lower-airway macrophage inflammation and neutrophil recruitment in SARS-CoV-2-infected rhesus macaques. Cell 184, 460-475 e421 (2021).
29. Zhang, Q. et al. Heparan sulfate assists SARS-CoV-2 in cell entry and can be targeted by approved drugs in vitro. Cell Discov 6, 80 (2020).
30. Riva, L. et al. Discovery of SARS-CoV-2 antiviral drugs through large-scale compound repurposing. Nature 586, 113-119 (2020).
31. Zhao. H., Mendenhall, M.& Deininger, M. W. Imatinib is not a potent anti-SARS-CoV-2 drug. Leukemia 34, 3085-3087 (2020).
32. Raghuvanshi, R. & Bharate, S. R. Recent Developments in the Use of Kinase Inhibitors for Management of Viral infections. J Med Chem 65, 893-921 (2022).
33. Drayman, N. et al. Masitinib is a broad coronavirus 3CL inhibitor that blocks replication of SARS-CoV-2. Science 373.931-936 (2021).
34. Corman, V. M. et al. Link of a ubiquitous human coronavirus to dromedary camels. Proc Natl Acad Sci USA 113, 9864-9869 (2016).
35. Lim, Y. X., Ng, Y. L., Tam, J. P. & Liu, D. X. Human Coronaviruses: A Review of Virus-Host Interactions. Diseases 4(3), 26 (2016).
36. Fehr, A. R. & Perlman, S. Coronaviruses: an overview of their replication and pathogenesis. Methods Mol Biol 1282, 1-23 (2015).
37. Seidel. S. A. et al. Microscale thermophoresis quantifies biomolecular interactions under previously challenging conditions. Methods 59, 301-315 (2013).
38. Willemsen, M. J. et at. MicroScale Thermophoresis: Interaction analysis and beyond. J Mol Structure 1077, 101-113 (2014).
39. Wrapp, D. et al. Cryo-EM structure of the 2019-nCoV spike in the prefusion conformation. Science 367, 1260-1263 (2020).
40. Chan, K. K. et al. Engineering human ACE2 to optimize binding to the spike protein of SARS coronavirus 2. Science 369, 1261-1265 (2020).
41. Klann, K. et at. Growth Factor Receptor Signaling Inhibition Prevents SARS-CoV-2 Replication. Mol Cell 80, 164-174 e164 (2020).
42. McCray, P. B., Jr. et al. Lethal infection of K18-hACE2 mice infected with severe acute respiratory syndrome coronavirus. J Virol 91, 813-821 (2007).
43. Oladunni, F. S. et al. Lethality of SARS-CoV-2 infection in K18 human angiotensin-converting enzyme 2 transgenic mice. Nat Commun 11, 6122 (2020).
44. Bao, L et al. The pathogenicity of SARS-CoV-2 in hACE2 transgenic mice. Nature 583, 830-833 (2020).
45. Harvey. W. T. et al. SARS-CoV-2 variants, spike mutations and immune escape. Nat Rev Microbiol 19, 409-424 (2021).
46. Forni, G., Mantovani, A.& COVID-19 Commission of Accademia Nazionale dei Lincei, R. m. COVID-19 vaccines: where we stand and challenges ahead. Cell Death Differ 28, 626-639 (2021).
47. Klann, K. et al. Growth Factor Receptor Signaling Inhibition Prevents SARS-CoV-2 Replication. Mol Cell 80, 164-174.e164 (2020).
48. Frampton, J. E. Vandetanib: in medullary thyroid cancer. Drugs 72, 1423-1436 (2012).
49. Wang, J. et al. Specific cytokines in the inflammatory cytokine storm of patients with COVID-19-associated acute respiratory distress syndrome and extrapulmonary multiple-organ dysfunction. Virol J18, 117 (2021).
50. Aid, M. et al. Vascular Disease and Thrombosis in SARS-CoV-2-infected Rhesus Macaques. Cell 183, 1354-1366 e1313 (2020).
51. Huang, C. et al. Clinical features of patients infected with 2019 novel coronavirus in Wuhan, China. Lancet 395, 497-506 (2020).
52. Herold, T. et al. Elevated levels of IL-6 and CRP predict the need for mechanical ventilation in COVID-19. J Allergy Clin Immunol 146, 128-136.e124 (2020).
53. Zhang, Q. et al. Inborn errors of type I IFN immunity in patients with life-threatening COVID-19. Science 370 (2020).
54. Xia, H. et al. Evasion of Type I Interferon by SARS-CoV-2. Cell Rep 33, 108234 (2020).
55. Yuen, C. K. et al. SARS-CoV-2 nsp13, nsp14, nsp15 and orf6 function as potent interferon antagonists. Emerg Microbes Infect 9, 1418-1428 (2020).
56. Molony, R. D. et al. Aging impairs both primary and secondary RIG-I signaling for interferon induction in human monocytes. Sci Signal 10 (2017).
57. Hu, R., Xia, C. Q., Butfiloski, E. & Clare-Salzler, M. Effect of high glucose on cytokine production by human peripheral blood immune cells and type I interferon signaling in monocytes: Implications for the role of hyperglycemia in the diabetes inflammatory process and host defense against infection. Clin Immunol 195, 139-148 (2018).
58. Chi, Y. et al. Serum Cytokine and Chemokine Profile in Relation to the Severity of Coronavirus Disease 2019 in China. J Infect Dis 222, 746-754 (2020).
59. Abers. M. S. et al. An immune-based biomarker signature is associated with mortality in COVID-19 patients. JCI Insight 6 (2021).
60. Sierra, B. et al. Association of Early Nasopharyngeal Immune Markers With COVID-19 Clinical Outcome: Predictive Value of CCL2/MCP-1. Open Forum Infect Dis 7, ofaa407 (2020).
61. Kaner, R. J. et al. Lung overexpression of the vascular endothelial growth factor gene induces pulmonary edema. Am J Respir Cell Mol Biol 22, 657-664 (2000).
62. Marti, H. H. & Risau, W. Systemic hypoxia changes the organ-specific distribution of vascular endothelial growth factor and its receptors. Proc Natl Acad Sci USA 95, 15809-15814 (1998).
63. Lee. C. G. et al. Vascular endothelial growth factor (VEGF) induces remodeling and enhances TH2-mediated sensitization and inflammation in the lung. Nat Md 10, 1095-1103 (2004).
64. Pang, J. et al. Efficacy and tolerability of bevacizumab in patients with severe Covid-19. Nat Commun 12, 814 (2021).
65. Weisberg, E. et al. Repurposing of Kinase Inhibitors for Treatment of COVID-19. Pharm Res 37, 167 (2020).
66. Sheahan, T. P. et al. Broad-spectrum antiviral GS-5734 inhibits both epidemic and zoonotic coronaviruses. Sci Transl Med 9 (2017).
67. Beigel, J. H. et al. Remdesivir for the Treatment of Covid-19—Final Report. N Engl J Med 383, 1813-1826 (2020).
68. Group, R. C. et al. Dexamethasone in Hospitalized Patients with Covid-19. N Engl J Med 384, 693-704 (2021).
69. Guimaraes, P. O. et al. Tofacitinib in Patients Hospitalized with Covid-19 Pneumonia. N Engl J Med 385, 406-415 (2021).
70. Kyriazopoulou, E. et al. Author Correction: Early treatment of COVID-19 with anakinra guided by soluble urokinase plasminogen receptor plasma levels: a double-blind, randomized controlled phase 3 trial. Nat Med 27, 1850 (2021).
71. Drayman, N. et al. Masitinib is a broad coronavirus 3CL inhibitor that blocks replication of SARS-CoV-2. Science 373, 931-936 (2021).
72. Martin, P. et al. Pharmacokinetics of vandetanib: three phase I studies in healthy subjects. Clin Ther 34, 221-237 (2012).
73. Jeong, W., Doroshow, J. H. & Kummar, S. United States Food and Drug Administration approved oral kinase inhibitors for the treatment of malignancies. Curr Probl Cancer 37, 110-144 (2013).
74. Prete, A. et al. Thyroid cancer and COVID-19: experience at one single thyroid disease referral center. Endocrine 72, 332-339 (2021).
75. Hou, Y. J. et al. SARS-CoV-2 Reverse Genetics Reveals a Variable Infection Gradient in the Respiratory Tract. Cell 182, 429-446 e414 (2020).
76. Yount, B., Denison, M. R., Weiss, S. R. & Baric, R. S. Systematic assembly of a full-length infectious cDNA of mouse hepatitis virus strain A59. J Viro 76, 11065-11078 (2(0)2).
77. Spearman, C. The method of ‘right and wrong cases’ (‘constant stimuli’) without Gauss's formulae. Brit J Psychol 2, 227-242 (1908).
78. Kärber, G. Beitrag zur kollektiven behandlung pharmakologischer reihenversuche. Archiv f Experiment Pathol u Pharmakol 162, 480-483 (1931).
79. Oladunni, F. S. et al. Lethality of SARS-CoV-2 infection in K18 human angiotensin-converting enzyme 2 transgenic mice. Nat Commun 11, 6122(2020).
80. Bao, L. et al. The pathogenicity of SARS-CoV-2 in hACE2 transgenic mice. Nature 583, 830-833 (2020).
81. Yinda, C. K. et al. K18-hACE2 mice develop respiratory disease resembling severe COVID-19. PLoS Pathog 17, e1009195 (2021).
82. Arce, V. M. & Costoya, J. A. SARS-CoV-2 infection in K18-ACE2 transgenic mice replicates human pulmonary disease in COVID-19. Cell Mol Immunol 18, 513-514 (2021).
83. Moreau, G. B. et al. Evaluation of K18-. Am J Trop Med Hvg 103, 1215-1219 (2020).
84. Winkler, E. S. et al. Publisher Correction: SARS-CoV-2 infection of human ACE2-transgenic mice causes severe lung inflammation and impaired function. Nat Immunol 21, 1470 (2020).
85. Zheng, J. et al. COVID-19 treatments and pathogenesis including anosmia in K18-hACE2 mice. Nature 589, 603-607 (2021).
86. Hsia, C. C., Hyde, D. M., Ochs, M., Weibel, E. R. & Structure, A.E.J.T.F.o.Q.A.o.L. An official research policy statement of the American Thoracic Society/European Respiratory Society: standards for quantitative assessment of lung structure. Am J Respir Crit Care Med 181, 394-418 (2010).
87. Nair, A. B., and Jacob, S. A. A simple practice guide for dose conversion between animals and humans. J. Basic Clin. Pharm. 7(2), 27-31 (2016).

It will be understood that various details of the presently disclosed subject matter may be changed without departing from the scope of the presently disclosed subject matter. Furthermore, the foregoing description is for the purpose of illustration only, and not for the purpose of limitation.

The invention claimed is:

1. A method of treating a lung condition in a subject in need of treatment thereof, the method comprising administering to the subject a therapeutically effective amount of vandetanib or a pharmaceutically acceptable salt thereof.

2. The method of claim 1, wherein the lung condition is characterized by a level of one or more cytokines that are elevated in the subject compared to a subject that does not have the lung condition.

3. The method of claim 1, wherein the lung condition is selected from the group consisting of coronavirus disease (COVID-19), bronchiopulmonary dysplasia, pulmonary fibrosis, and chemical exposure.

4. The method of claim 1, wherein the subject is a mammalian subject.

5. The method of claim 4, wherein the subject is a human subject.

6. The method of claim 1, wherein the administration reduces a level of one or more of interleukin-6 (IL-6), interleukin-10 (IL-10), and tumor necrosis factor alpha (TNF-α) in the subject.

7. The method of claim 1, wherein the administration increases a level of interferon 1β (IFN-β) in the subject.

8. The method of claim 1, wherein the administration reduces a level of one or more chemokine (C—C motif) ligand (CCL) in the subject.

9. The method of claim 8, wherein the one or more CCL is selected from the group consisting of CCL2, CCL3, and CCL4.

10. A method of treating coronavirus disease (COVID-19) in a subject in need of treatment thereof, the method comprising administering to the subject a therapeutically effective amount of vandetanib or a pharmaceutically acceptable salt thereof.

11. The method of claim 10, wherein the COVID-19 is characterized by a level of one or more cytokines that are elevated in the subject compared to a subject that does not have the lung condition.

12. The method of claim 10, wherein the subject is a mammalian subject.

13. The method of claim 12, wherein the subject is a human subject.

14. The method of claim 13, wherein the subject is a hospitalized subject and/or subject receiving supplemental oxygen.

15. The method of claim 10, wherein the administration reduces a level of one or more of interleukin-6 (IL-6), interleukin-10 (IL-10), and tumor necrosis factor alpha (TNF-α) in the subject.

16. The method of claim 10, wherein the administration increases a level of interferon 1β (IFN-β) in the subject.

17. The method of claim 10, wherein the administration reduces a level of one or more chemokine (C—C motif) ligand (CCL) in the subject.

18. The method of claim 17, wherein the one or more CCL is selected from the group consisting of CCL2, CCL3, and CCL4.

19. (canceled)

20. (canceled)

21. (canceled)
